SEQUENCE LISTING (9 sequences)

SEQ ID NO: 1 - DNA, 1281 bp, Homo sapiens
  human SMAD7
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 2 - DNA, 20 bp, Artificial Sequence
  SMAD7 antisense oligonucleotide
gtcgccccttctccccgcag

SEQ ID NO: 3 - DNA, 21 bp, Artificial Sequence
  SMAD7 antisense oligonucleotide
gtcgccccttctccccgcagc

SEQ ID NO: 4 - DNA, 20 bp, Artificial Sequence
  SMAD7 antisense oligonucleotide
  modified_base (3)..(3): n = 5-methyl 2′-deoxycytidine
  modified_base (16)..(16): n = 5-methyl 2′-deoxycytidine
gtngccccttctcccngcag

SEQ ID NO: 5 - DNA, 21 bp, Artificial Sequence
  SMAD7 antisense oligonucleotide
  modified_base (3)..(3): n = 5-methyl 2′-deoxycytidine
  modified_base (16)..(16): n = 5-methyl 2′-deoxycytidine
gtngccccttctcccngcagc

SEQ ID NO: 6 - DNA, 20 bp, Artificial Sequence
  SMAD7 antisense oligonucleotide
  modified_base (3)..(3): cytosine, 5-methylcytosine, 2-O-methylcytosine
  modified_base (4)..(4): guanine, 5-methylguanine, 2-O-methylguanine
  modified_base (16)..(16): cytosine, 5-methylcytosine, 2-O-methylcytosine
  modified_base (17)..(17): guanine, 5-methylguanine, 2-O-methylguanine
gtnnccccttctcccnncag

SEQ ID NO: 7 - DNA, 21 bp, Artificial Sequence
  SMAD7 antisense oligonucleotide
  modified_base (3)..(3): n = 5-methyl-2′-deoxycytidine
  modified_base (16)..(16): n = 5-methyl-2′-deoxycytidine
gtngccccttctcccngcagc

SEQ ID NO: 8 - DNA, 20 bp, Artificial Sequence
  an antisense oligonucleotide phosphorothioate against SMAD7
  modified_base (3)..(3): n = 5-methyl-2′-deoxycytidine
  modified_base (16)..(16): n = 5-methyl-2′-deoxycytidine
gtngccccttctcccngcag

SEQ ID NO: 9 - DNA, 21 bp, Artificial Sequence
  an antisense oligonucleotide phosphorothioate against SMAD7
  modified_base (3)..(3): n = 5-methyl-2′-deoxycytidine
  modified_base (16)..(16): n = 5-methyl-2′-deoxycytidine
gtngccccttctcccngcagc

(19) United States
(12) Patent Application Publication
(10) Pub. No.: US 2017/0247695 A1
(43) Pub. Date: Aug. 31, 2017

(54) ISOTOPOLOGUES OF SMAD7 ANTISENSE OLIGONUCLEOTIDES

(71) Applicant: CELGENE CORPORATION, Summit, NJ (US)

(72) Inventor: Peter Anthony CICALA, Summit, NJ (US)

(21) Appl. No.: 15/519,502

(22) PCT Filed: Oct. 17, 2014
(86) PCT No.: PCT/IB2014/065421

§ 371 Date: Apr. 14, 2017

(51) Int. Cl. C12N 15/113

(52) U.S. Cl. CPC C12N 15/113; C12N 2310/11; C12N 2310/315; C12N 2310/3341

(57) ABSTRACT

This disclosure relates generally to deuterated isotopologues of SMAD7 antisense oligonucleotides, pharmaceutical compositions containing the same, and methods of using the same.

### FIELD

This disclosure relates generally to isotopologues of SMAD7 antisense oligonucleotides, pharmaceutical compositions containing the same, and methods of using the same.

### BACKGROUND

Recent studies have demonstrated an involvement of the tumor growth factor beta (TGF-β) signaling pathway in inflammatory diseases. Specifically, SMAD7, an intracellular protein binding to TGF-β receptor and inhibiting TGF-β receptor signaling, has emerged as a drug target candidate for inflammatory disease indications, such as inflammatory bowel diseases (IBD).

IBD is a chronic inflammatory disorder of the gastrointestinal tract. The two most common forms of IBD are Crohn's disease (CD) and ulcerative colitis (UC). Although CD primarily affects the terminal ileum and right colon can affect the entire gastrointestinal tract, it primarily affects the ileum (the distal or lower portion of the small intestine) and the large intestine. UC primarily affects the colon and the rectum. Current treatments for both CD and UC include aminoslicylates, antibiotics, corticosteroid, immunosuppressants and tumor necrosis factor alpha (TNFα) antagonists. However, patient responses to these treatments can vary with disease severity and many current treatments are associated with undesirable side effects. Thus there is a need to identify new treatments for IBD, including CD and UC.

A SMAD7 antisense oligonucleotide was shown to down-regulate, prevent and treat CD-like symptoms in mice and a Phase I clinical study suggested clinical benefits in human CD patients resulting from the administration of a SMAD7 antisense oligonucleotide.

### SUMMARY

Provided herein are isotopologues of SMAD7 antisense oligonucleotides, pharmaceutical compositions containing the same, and methods of using the same.

In one aspect, provided herein are a deuterated SMAD7 antisense oligonucleotide, including a plurality of hydrogens (H), wherein one or more hydrogens of the plurality of hydrogens are replaced by deuterium (D).

In some embodiments, the one or more hydrogen replaced by deuterium is enriched in deuterium to more than 0.02%, more than 0.03%, more than 0.1%, more than 0.3%, more than 1%, more than 3%, more than 10%, more than 15%, more than 20%, more than 25%, more 30%, more than 35%, more than 40%, more than 45%, more than 50%, more than 55%, more than 60%, more than 65%, more than 70%, more than 75%, more than 80%, more than 85%, more than 90%, more than 95%, more than 98% or more than 99%.

In some embodiments, at least 1%, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of hydrogens of the plurality of hydrogens are replaced with deuterium.

In some embodiments, the deuterated SMAD7 antisense oligonucleotide further includes a plurality of nucleotides, wherein at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of nucleotides of the plurality of nucleotides are partially or fully deuterated.

In some embodiments, the deuterated SMAD7 antisense oligonucleotide further includes a plurality of nucleobases, wherein one or more nucleobases of the plurality of nucleobases are deuterated.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of nucleobases are partially or fully deuterated.

In some embodiments, the deuterated SMAD7 antisense oligonucleotide further includes a plurality of riboses or deoxyriboses, wherein one or more riboses or deoxyriboses of the plurality of riboses or deoxyriboses are deuterated.

In some embodiments, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of riboses or deoxyriboses of the plurality of deoxyriboses are partially or fully deuterated.

In some embodiments, the deuterated SMAD7 antisense oligonucleotide targets a region of 10 or more, 12 or more, 14 or more, 16 or more, 18 or more, 20 or more, 22 or more, 24 or more, 26 or more, 28 or more or 30 or more consecutive nucleotides of human SMAD7 (SEQ ID NO: 1).

In some embodiments, the deuterated SMAD7 antisense oligonucleotide targets region 108-128 of human SMAD7 (SEQ ID NO: 1) (CDS of NM_005904.3).

In some embodiments, the deuterated SMAD7 antisense oligonucleotide targets nucleotides 403, 233, 294, 295, 296, 298, 299 or 533 of human SMAD7 (SEQ ID NO: 1).

In some embodiments, the deuterated SMAD7 antisense oligonucleotide includes the nucleotide sequence of SEQ ID NO: 2 (5′-GTCGCCCCTTCTCCCCGCAG-3′).

In some embodiments, the deuterated SMAD7 antisense oligonucleotide includes the nucleotide sequence of SEQ ID NO: 3 (5′-GTCGCCCCTTCTCCCCGCAGC-3′).

In some embodiments, at least one internucleoside linkage is a phosphorothioate linkage.

In some embodiments, all internucleoside linkages are phosphorothioate linkages.

In some embodiments, 2′-deoxyribonucleotides are replaced by corresponding ribonucleotides.

In some embodiments, the deuterated SMAD7 antisense oligonucleotide is an antisense oligonucleotide phosphorothioate against SMAD7 including the following sequence: 5′-GTXGCCCCTTCTCCCXGCAG-3′ (SEQ ID NO: 8) wherein X is 5-methyl-2′-deoxycytidine and wherein all internucleotide linkages are phosphorothioate linkages.

In some embodiments, the deuterated SMAD7 antisense oligonucleotide is an antisense oligonucleotide phosphorothioate against SMAD7 including the following sequence: 5′-GTXGCCCCTTCTCCCXGCAGC-3′ (SEQ ID NO: 9) wherein X is 5-methyl-2′-deoxycytidine and wherein the internucleotide linkages are phosphorothioate linkages.

In some embodiments, the deuterated SMAD7 antisense oligonucleotide comprises Formula (I). The following structure of Formula (I) is drawn over four pages:

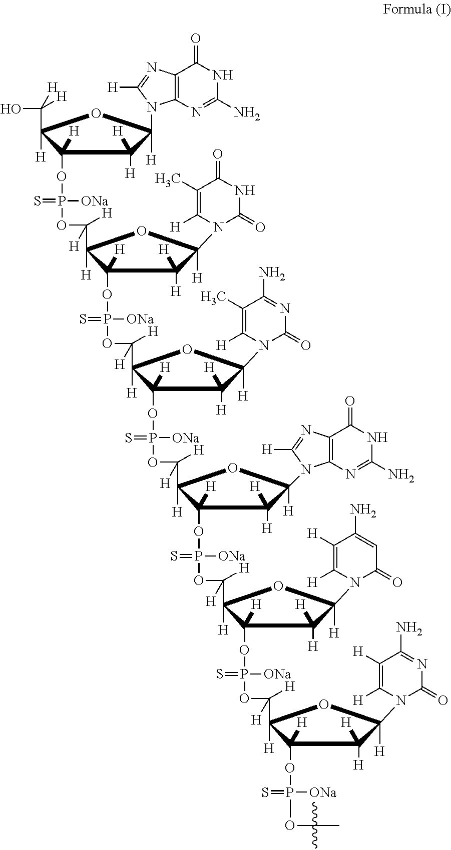

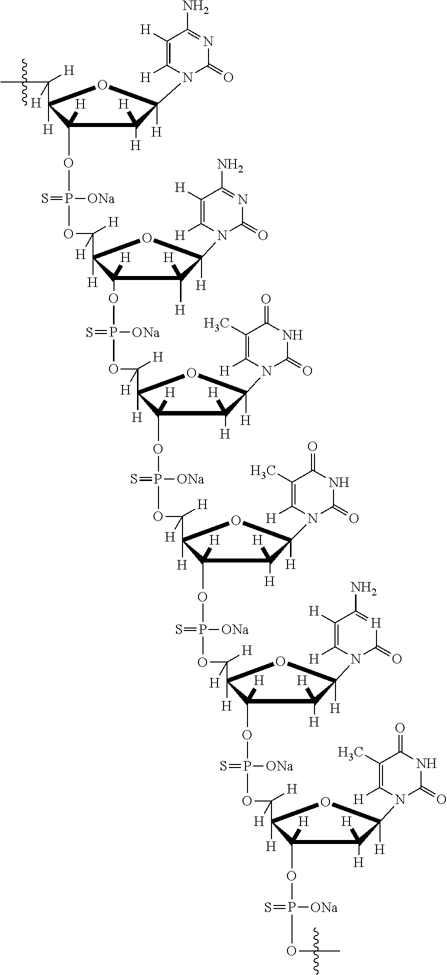

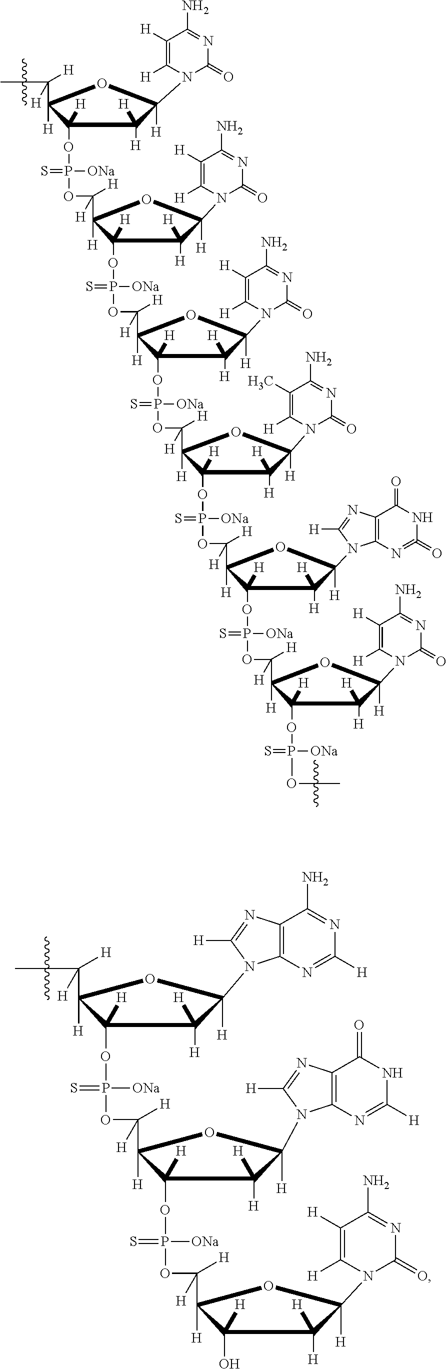

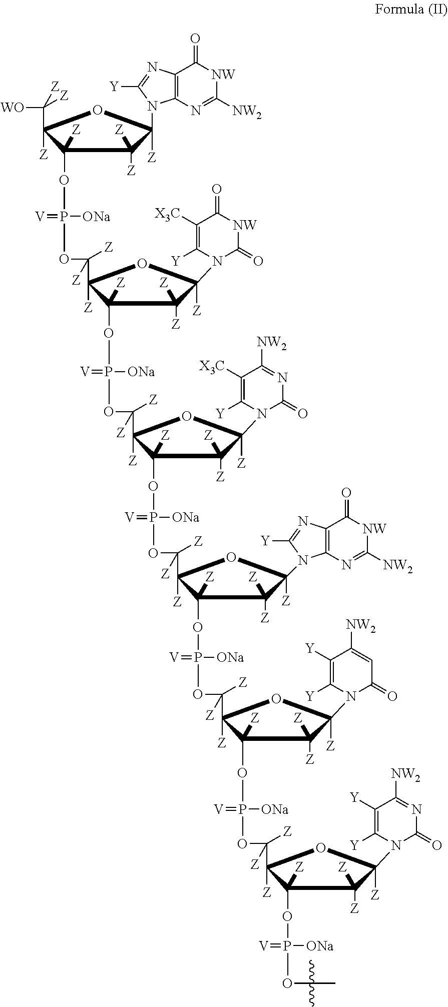

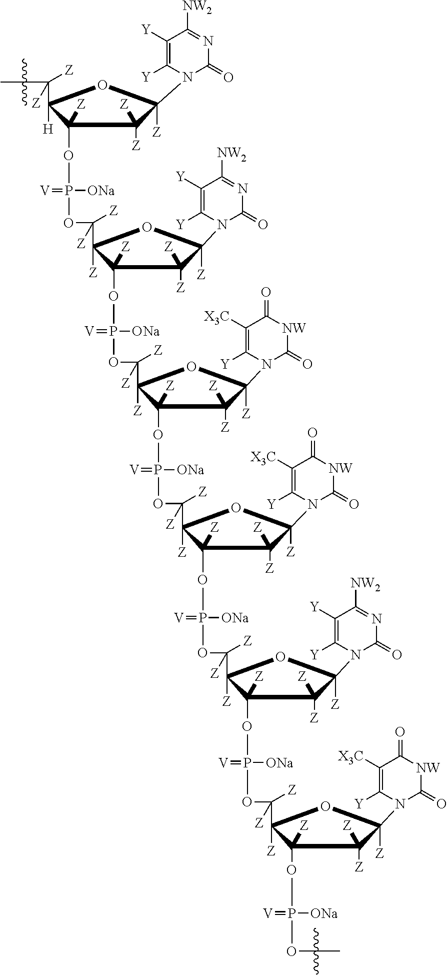

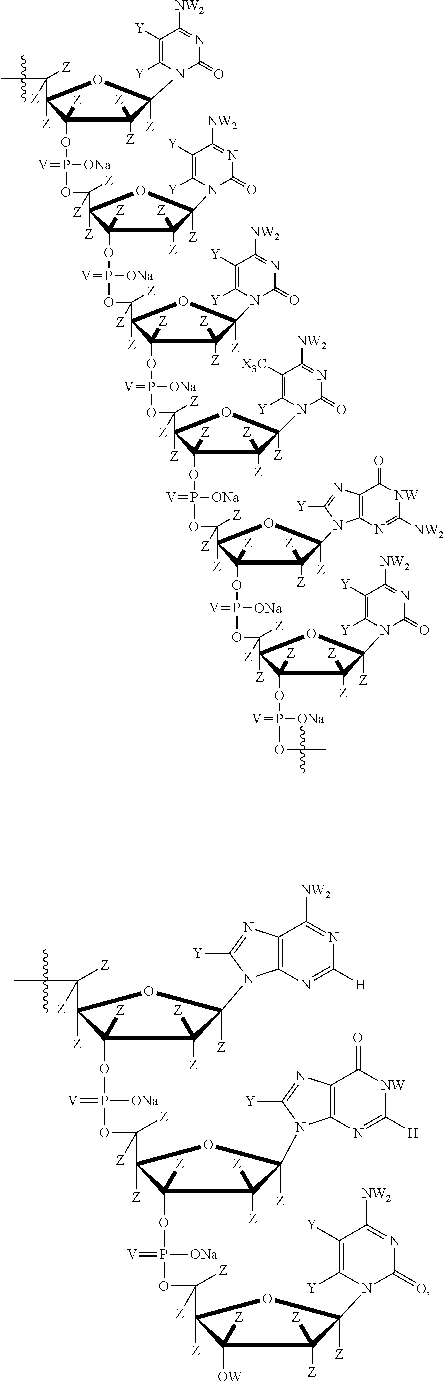

or a pharmaceutically acceptable salt or solvent thereof.

In some embodiments, the deuterated SMAD7 antisense oligonucleotide includes a plurality of deuterated SMAD7 antisense oligonucleotides.

In some embodiments, the deuterated SMAD7 antisense oligonucleotide is a pharmaceutically acceptable salt or solvent.

In some embodiments, the deuterated SMAD7 antisense oligonucleotide comprises a SMAD7 antisense oligonucleotide of Formula (II). The following structure of Formula (II) is drawn over four pages:

or a pharmaceutically acceptable salt or solvate thereof, wherein V is oxygen or sulfur and W, X, Y, and Z are hydrogen or deuterium.

In some embodiments, V is oxygen.

In some embodiments, V is sulfur.

In some embodiments, more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, or more than 97% of W are deuterium.

In some embodiments, more than 1%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of X are deuterium.

In some embodiments, more than 1%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of Y are deuterium.

In some embodiments, more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 97%, more than 98%, or more than 99% of Z are deuterium

In some embodiments, about the same fraction of W, X, Y and Z in Formula (II) are D.

In some embodiments, the fractions of W, X, Y and Z in Formula (II) that are D are different.

In some embodiments, the relative sizes of the fractions of W, X, Y and Z of Formula (II) that are D has a relationship according to Table 1.

In some embodiments, the degree of deuteration of any one deuterated W of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

In some embodiments, the degree of deuteration of any one deuterated W of Formula (II) is 100%.

In some embodiments, the degree of deuteration of different deuterated W of Formula (II) is about the same

In some embodiments, the degree of deuteration of different deuterated W of Formula (II) is different.

In some embodiments, the degree of deuteration of only one deuterated X of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

In some embodiments, the degree of deuteration of any one deuterated X of Formula (II) is 100%.

In some embodiments, the degree of deuteration of only one deuterated X of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

In some embodiments, the degree of deuteration of different deuterated X of Formula (II) is about the same.

In some embodiments, the degree of deuteration of different deuterated X of Formula (II) is different.

In some embodiments, the degree of deuteration of only one deuterated Y of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

In some embodiments, the degree of deuteration of any one deuterated Y of Formula (II) is 100%.

In some embodiments, the degree of deuteration of only one deuterated Z of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

In some embodiments, the degree of deuteration of any one deuterated Z of Formula (II) is 100%.

In some embodiments, the degree of deuteration of different deuterated Z of Formula (II) is about the same.

In some embodiments, the degree of deuteration of different deuterated Z of Formula (II) is different.

In some embodiments, the average, median, or mean degree of deuteration for the deuterated W, X, Y and Z in Formula (II) is be about the same.

In some embodiments, the average, median or mean degree of deuteration is different for deuterated W, X, Y and Z of Formula (II).

In some embodiments, the relative average, median or mean degrees of deuteration of deuterated W, X, Y, and Z of Formula (II) has a relationship according to Table 1.

In some embodiments, the deuterated SMAD7 antisense oligonucleotide is a pharmaceutically acceptable salt.

In some embodiments, the deuterated SMAD7 antisense oligonucleotide is a sodium salt.

In some embodiments, replacing the one or more hydrogens by deuterium (D) creates one or more chiral centers.

In some embodiments, the one or more chiral centers comprise one or more C2′ or C5′ atoms in a ribose or deoxyribose in the deuterated SMAD7 antisense oligonucleotide.

In some embodiments, more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 75%, more than 80%, more than 85%, more than 90% or more than 95% or C2′ and/or C5′ of a D-ribose or D-deoxyribose in a deuterated SMAD7 antisense oligonucleotide are chiral centers.

In some embodiments, the deuterated SMAD7 antisense oligonucleotide includes a racemic mixture of deuterated SMAD7 antisense oligonucleotides.

In some embodiments, the deuterated SMAD7 antisense oligoucleotide includes an enantiomeric excess.

In some embodiments, the deuterated SMAD7 antisense oligonucleotide includes a mixture of diastereomers.

In some embodiments, the deuterated SMAD7 antisense oligonucleotide includes a SMAD7 antisense oligonucleotide of Formula (III). The following structure of Formula (III) is drawn over four pages:

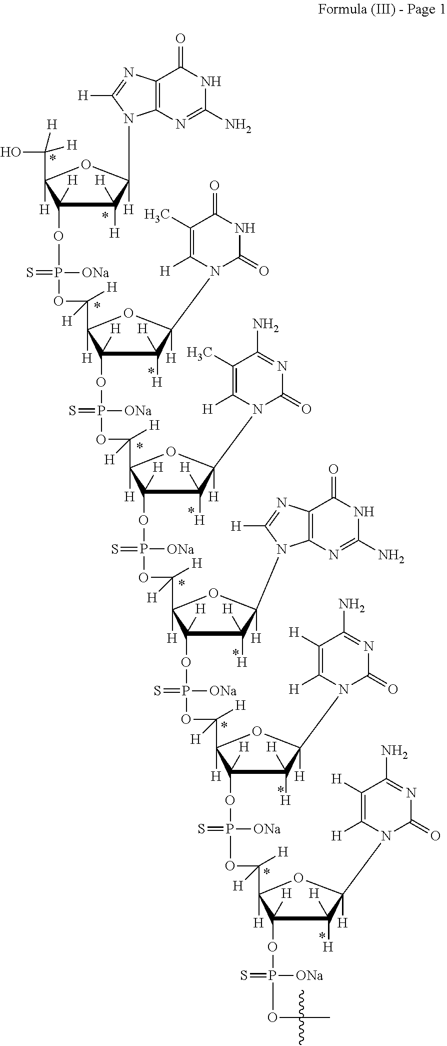

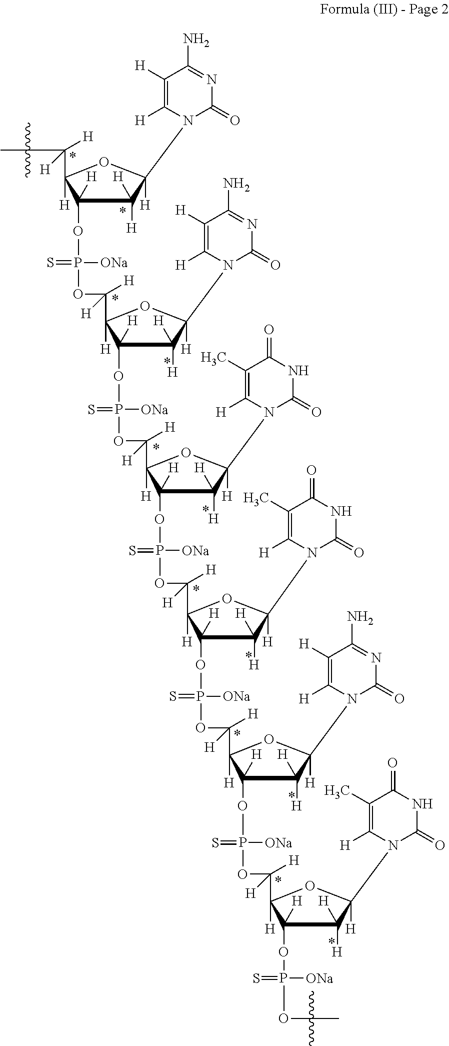

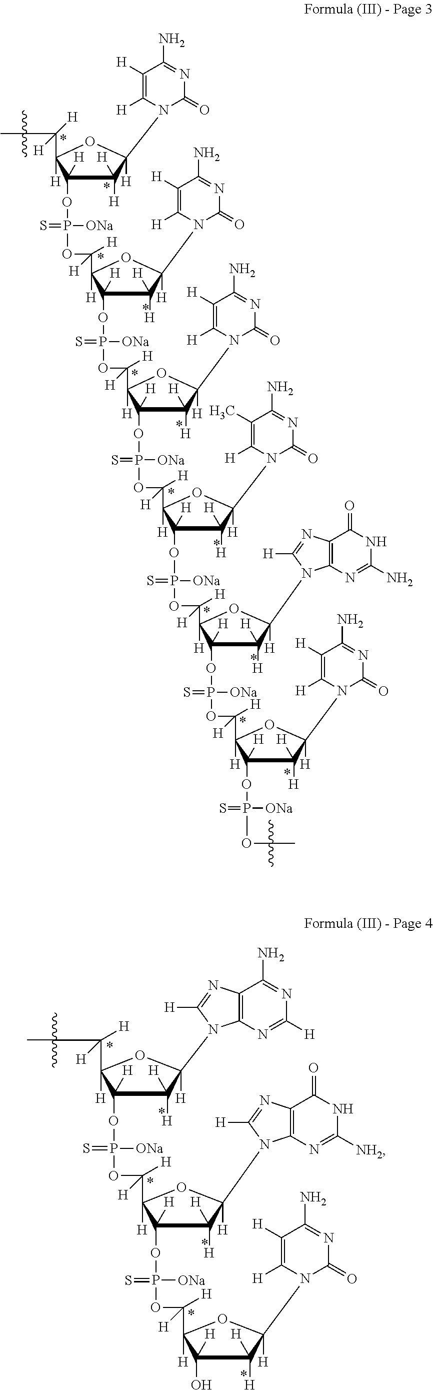

or a pharmaceutically acceptable salt or solvate thereof, wherein one or more H are replaced by D.

In some embodiments, essentially all D in the deuterated SMAD7 antisense oligonucleotide are present in one or more nucleotides of interest.

In some embodiments, essentially all D in the deuterated SMAD7 antisense oligonucleotide are present in one or more nucleobases of interest.

In some embodiments, the nucleobase includes a purine.

In some embodiments, the nucleobase includes a pyrimidine.

In some embodiments, the nucleobases of interest are selected from adenine, guanine, cytosine, thymine or uracil.

In some embodiments, essentially all D in the deuterated SMAD7 antisense oligonucleotide are present in one or more sugar moieties of interest.

In some embodiments, the one or more sugar moieties of interest are a ribose or deoxyribose moiety.

In some embodiments, one or more nucleotides of interest in the deuterated SMAD7 antisense oligonucleotide includes one or more D.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of the nucleotides of interest include one or more D.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 nucleotides of interest include one or more D.

In some embodiments, one or more nucleotides of interest each comprise at least 1, at least 2, at least 3, at least 4, at least 5, at least 6, at least 7, at least 8, at least 9, at least 10, at least 11 or at least 12 D.

In some embodiments, each of the one or more nucleotides of interest include the same number of D.

In some embodiments, two or more nucleotides of interest include different numbers of D.

In some embodiments, one or more nucleobases of interest include one or more D.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of nucleobases of interest include one or more D.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 nucleotides of interest include one or more D.

In some embodiments, each of the one or more nucleobases of interest include at least 1, at least 2, at least 3, at least 4, or at least 5 D.

In some embodiments, each of the nucleobases of interest include the same number of D.

In some embodiments, two or more nucleobases of interest include different numbers of D.

In some embodiments, the one or more nucleobases of interest include a purine.

In some embodiments, the purine is an adenine or a guanine.

In some embodiments, the one or more nucleobases of interest include a pyrimidine.

In some embodiments, the pyrimidine is a cytosine, thymine or uracil.

In some embodiments, one or more sugar moieties of interest include one or more D.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of sugar moieties of interest include one or more D.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 sugar moieties of interest include one or more D.

In some embodiments, each of the one or more sugar moieties of interest include at least 1, at least 2, at least 3, at least 4, or at least 5 D.

In some embodiments, each of the sugar moieties of interest includes the same number of D.

In some embodiments, two or more sugar moieties of interest include different numbers of D.

In some embodiments, the one or more sugar moieties of interest include a ribose or deoxyribose.

In another aspect, provided herein is a pharmaceutical composition including a deuterated SMAD7 antisense oligonucleotide provided herein and a pharmaceutically acceptable adjuvant and/or excipient.

In some embodiments, the pharmaceutical composition is an oral pharmaceutical composition.

In another aspect, provided herein is a method of treating inflammatory bowel disease (IBD), including administering to a patient in need thereof an effective amount of a deuterated SMAD7 antisense oligonucleotide provided herein, wherein the deuterated SMAD7 antisense oligonucleotide is effective to treat IBD.

In some embodiments, IBD is Crohn's disease.

In some embodiments, IBD is ulcerative colitis.

In some embodiments, the deuterated SMAD7 antisense oligonucleotide is administered orally.

### DETAILED DESCRIPTION

The term “isotopically enriched” refers to an atom having an isotopic composition other than the natural isotopic composition of that atom. “Isotopically enriched” can also refer to a compound containing at least one atom having an isotopic composition other than the natural isotopic composition of that atom. As used herein, an “isotopologue” is an isotopically enriched compound.

The term “isotopic enrichment” refers to the percentage of incorporation of an amount of a specific isotope at a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

With regard to the compounds provided herein, when a particular atomic position is designated as having deuterium or “D,” it is understood that the abundance of deuterium at that position is substantially greater than the natural abundance of deuterium, which is about 0.015%.

In one aspect, provided herein are isotopically enriched SMAD7 antisense oligonucleotides that have a plurality of hydrogens, wherein one or more hydrogens of the plurality of hydrogens are replaced by deuterium.

The present disclosure provides isotopologues of anti-SMAD7 agents, pharmaceutical compositions containing such isotopologues, and uses for isotopologues of anti-SMAD7 agents, in particular as therapeutic agents for the treatment of a chronic inflammatory bowel disease (IBD), such as Crohn's disease (CD) or ulcerative colitis (UC).

The isotopologues of anti-SMAD7 agents provided herein can include, e.g., isotopologues of SMAD7 antisense oligonucleotides or isotopologues of SMAD7 small interfering RNA (siRNA, RNAi), or the like.

The isotopologues of anti-SMAD7 agents provided herein are isotopically enriched for one or more isotopes, including, e.g., without limitation, deuterium (D), tritium (T), ¹³C, ³³S, ³⁴S, ³⁶S, ¹⁵N, ¹⁷O and ³¹P. In addition, compositions containing combinations of two or more isotopes are also contemplated. Furthermore, isotope-depleted compounds which lack the natural abundance distribution are contemplated. For example compounds composed of 100% ¹H, ¹²C or ¹⁶O, etc.

This disclosure is based, in part, on the realization that the isotopic enrichment (e.g., deuteration) of therapeutic agents, such as antisense oligonucleotides, can have beneficial effects on pharmacokinetic, pharmacodynamic, and toxicity profiles of the therapeutic agents, e.g., by increasing their in vivo half-lives.

For example, and without wishing to be bound by any particular theory, it is believed that isotope enrichment (e.g., deuteration) of certain H-atoms can reduce the in vivo oxidation of nucleobases and thereby increase the effective half-life of isotopically enriched SMAD7 antisense oligonucleotides. Especially, the nucleobases adenine and guanine are believed to be prone to in vivo oxidation. The oxidation of nucleobases can prevent H-bridge formation between SMAD7 antisense oligonucleotides and their SMAD7 target sequences and inactivate the SMAD7 antisense oligonucleotides. Isotopic enrichment (e.g., deuteration) of nucleobase protons can slow down and reduce the nucleobases' in vivo oxidation, and increase the efficacy and effective in vivo half-life of SMAD7 antisense oligonucleotides. In vivo oxidation commonly occurs in connection with inflammation and inflammatory diseases, such as IBD. In inflammation, in vivo oxidation is frequently mediated by reactive oxygen species (ROS), the elevated production of which is induced by inflammatory cytokines and chemokines, including tumor necrosis factor alpha (TNFα), interleukin 1 (IL1), interleukin 6 (IL6) or interleukin 8 (IL8).

In one aspect, provided herein are isotopically enriched SMAD7 antisense oligonucleotides or siRNAs. In some embodiments, the isotopically enriched SMAD7 antisense oligonucleotides or siRNAs are deuterated. In some embodiments, the deuterated SMAD7 antisense oligonucleotides or siRNAs have a plurality of hydrogens (H), whereby one or more H of the plurality of Hs are replaced by D.

### SMAD7 Antisense Oligonucleotides

Antisense oligonucleotides are short synthetic oligonucleotide sequences complementary to the messenger RNA (mRNA), which encodes for the target protein (e.g., SMAD7). Antisense oligonucleotide sequences hybridize to the mRNA producing a double-strand hybrid that can lead to the activation of ubiquitary catalytic enzymes, such as RNase H, which degrades DNA/RNA hybrid strands, thus preventing protein translation. Without being bound by theory, an antisense oligonucleotide provided herein can hybridize to its target sequence as RNA or DNA. Thus, even if a DNA sequence is provided as target, the corresponding RNA sequence (including uracil instead of thymine) is included.

The isotopically enriched SMAD7 antisense oligonucleotides provided herein specifically target SMAD7 from any one mammalian organism. Such mammalian organisms include, e.g., without limitation, humans, primates (e.g., monkeys, chimpanzees, orangutans, and gorillas), cats, dogs, rabbits, farm animals (e.g., cows, horses, goats, sheep, pigs), and rodents (e.g., mice, rats, hamsters, and guinea pigs).

The isotopically enriched SMAD7 antisense oligonucleotides can target any one region of SMAD7, including any translated region or any untranslated region. Any 8 or more, 10 or more, 12 or more, 14 or more, 16 or more, 18 or more, 20 or more, 22 or more, 24 or more, 26 or more, 28 or more or 30 or more consecutive nucleotides of SMAD7 can be targeted by the isotopically enriched SMAD7 antisense oligonucleotides.

In some embodiments, the isotopically enriched SMAD7 antisense oligonucleotides can target a region in human SMAD7. In some embodiments, the isotopically enriched SMAD7 antisense oligonucleotides can target a region of 8 or more, 10 or more, 12 or more, 14 or more, 16 or more, 18 or more, 20 or more, 22 or more, 24 or more, 26 or more, 28 or more or 30 or more consecutive nucleotides of human SMAD7. In some embodiments, the isotopically enriched SMAD7 antisense oligonucleotides can target a region in a human SMAD7 including the nucleic acid sequence of SEQ ID NO: 1, or the corresponding RNA sequence.

SEQ ID No: 1 (Coding Sequence: CDS (288-1568) of NM_005904.3; Homo sapiens SMAD family member 7 (SMAD7), transcript variant 1, mRNA) (region 108-128 underlined):

ATG TTCAGGACCA AACGATCTGC GCTCGTCCGG CGTCTCTGGA
GGAGCCGTGC GCCCGGCGGC GAGGACGAGG AGGAGGGCGC
AGGGGGAGGT GGAGGAGGAG GCGA [symbol] [symbol] [symbol]
GGACA GCCGAGCGCA TGGGGCCGGT GGCGGCGGCC
CGGGCAGGGC TGGATGCTGC CTGGGCAAGG CGGTGCGAGG
TGCCAAAGGT CACCACCATC CCCACCCGCC AGCCGCGGGC
GCCGGCGCGG CCGGGGGCGC CGAGGCGGAT CTGAAGGCGC
TCACGCACTC GGTGCTCAAG AAACTGAAGG AGCGGCAGCT
GGAGCTGCTG CTCCAGGCCG TGGAGTCCCG CGGCGGGACG
CGCACCGCGT GCCTCCTGCT GCCCGGCCGC CTGGACTGCA
GGCTGGGCCC GGGGGCGCCC GCCGGCGCGC AGCCTGCGCA
GCCGCCCTCG TCCTACTCGC TCCCCCTCCT GCTGTGCAAA
GTGTTCAGGT GGCCGGATCT CAGGCATTCC TCGGAAGTCA
AGAGGCTGTG TTGCTGTGAA TCTTACGGGA AGATCAACCC
CGAGCTGGTG TGCTGCAACC CCCATCACCT TAGCCGACTC
TGCGAACTAG AGTCTCCCCC CCCTCCTTAC TCCAGATACC
CGATGGATTT TCTCAAACCA ACTGCAGACT GTCCAGATGC
TGTGCCTTCC TCCGCTGAAA CAGGGGGAAC GAATTATCTG
GCCCCTGGGG GGCTTTCAGA TTCCCAACTT CTTCTGGAGC
CTGGGGATCG GTCACACTGG TGCGTGGTGG CATACTGGGA
GGAGAAGACG AGAGTGGGGA GGCTCTACTG TGTCCAGGAG
CCCTCTCTGG ATATCTTCTA TGATCTACCT CAGGGGAATG
GCTTTTGCCT CGGACAGCTC AATTCGGACA ACAAGAGTCA
GCTGGTGCAG AAGGTGCGGA GCAAAATCGG CTGCGGCATC
CAGCTGACGC GGGAGGTGGA TGGTGTGTGG GTGTACAACC
GCAGCAGTTA CCCCATCTTC ATCAAGTCCG CCACACTGGA
CAACCCGGAC TCCAGGACGC TGTTGGTACA CAAGGTGTTC
CCCGGTTTCT CCATCAAGGC TTTCGACTAC GAGAAGGCGT
ACAGCCTGCA GCGGCCCAAT GACCACGAGT TTATGCAGCA
GCCGTGGACG GGCTTTACCG TGCAGATCAG CTTTGTGAAG
GGCTGGGGCC AGTGCTACAC CCGCCAGTTC ATCAGCAGCT
GCCCGTGCTG GCTAGAGGTC ATCTTCAACA GCCGGTAG

In some embodiments, the isotopically enriched SMAD7 antisense oligonucleotides target region 108-128 of the human SMAD7 including the nucleic acid sequence of SEQ ID NO:1, or the corresponding RNA sequence.

In some embodiments, the isotopically enriched SMAD7 antisense oligonucleotides target nucleotides 403, 233, 294, 295, 296, 298, 299 or 533 of the human SMAD7 including the nucleic acid sequence of SEQ ID NO: 1, or the corresponding RNA sequence.

In some embodiments, the isotopically enriched SMAD7 antisense oligonucleotides include the nucleotide sequence of SEQ ID NO: 2 (5′-GTCGCCCCTTCTCCCCGCAG-3′).

In some embodiments, the isotopically enriched SMAD7 antisense oligonucleotides include the nucleotide sequence of SEQ ID NO: 3 (5′-GTCGCCCCTTCTCCCCGCAGC-3′).

The isotopically enriched SMAD7 antisense olignucleotides provided herein can include naturally occurring nucleobases, sugars, and covalent internucleotide (backbone) linkages, as well as non-naturally occurring portions. For example, the isotopically enriched SMAD7 antisense oligonucleotides can include a mixed-backbone, e.g., including one or more phosphorothioate linkages. In some embodiments, the isotopically enriched SMAD7 antisense oligonucleotides can have one or more cytosine residues replaced by 5-methylcytosine. In some embodiments the one or more cytosine residues form part of a CpG pair.

In some embodiments, the isotopically enriched SMAD7 antisense oligonucleotides can include artificial nucleotides, such as deoxycytidine and/or 5-methyl 2′-deoxycytidine, including, but not limited to, 5-methyl-2′-deoxycytidine 5′-monophosphate and 5-methyl-2′-deoxycytidine 5′-monophosphorothioate.

In some embodiments, the isotopically enriched SMAD7 antisense oligonucleotides include the nucleic acid sequence of SEQ ID NO:4 (5′-GTXGCCCCTTCTCCCXGCAG-3)′, wherein X is 5-methyl 2′-deoxycytidine.

In some embodiments, the isotopically enriched SMAD7 antisense oligonucleotides include the nucleic acid sequence of SEQ ID NO:5 (5′-GTXGCCCCTTCTCCCXGCAGC-3′), wherein X is 5-methyl 2′-deoxycytidine.

In some embodiments, the isotopically enriched SMAD7 antisense oligonucleotides include the nucleic acid sequence of SEQ ID NO: 6 (5′-GTXYCCCCTTCTCCCXYCAG-3′), whereby X is a nucleotide including a nitrogenous base selected from the group consisting of cytosine and 5-methylcytosine nucleoside or a 2′-O-methylcytosine nucleoside, and wherein Y is a nucleotide including a nitrogenous base selected from the group consisting of guanine and 5-methylguanine or a 2′-O-methylguanine nucleoside, optionally provided that at least one of the nucleotides X or Y comprises a methylated nitrogenous base.

In some embodiments, the isotopically enriched SMAD7 antisense oligonucleotides include the nucleic acid sequence of SEQ ID NO: 7: (5′-GTC*GCC CCT TCT CCC C*GC AGC-3′), whereby C* represents 5-methyl-2′-deoxycytidine. In some embodiments, at least one of the internucleotide linkages of the isotopically enriched SMAD7 antisense oligonucleotide is an O,O-linked phosphorothioate. In some embodiments, all of the internucleotide linkages of the isotopically enriched SMAD7 antisense oligonucleotide can be O,O-linked phosphorothioates. In some embodiments, the isotopically enriched SMAD7 antisense oligonucleotide is a SMAD7 antisense oligonucleotide comprising a nucleotide sequence of SEQ ID NO: 5, wherein each of the 20 internucleotide linkages is an O,O-linked phosphorothioate linkage.

In some embodiments, the isotopically enriched SMAD7 antisense oligonucleotides include at least one internucleoside linkage, which is a phosphate linkage, e.g., a monophosphate linkage.

In some embodiments, the isotopically enriched SMAD7 antisense oligonucleotides include at least one internucleoside linkage, which is a phosphorothioate linkage. In some embodiments, the isotopically enriched SMAD7 antisense oligonucleotides include at least 2, 3, 4, 5, 6, 7, 8, 9, 10, 11, 12, 13, 14, 15, 16, 17, 18, 19, 20, 21, 22, 23, 24, 25, or more phosphorothioate linkages. In some embodiments, at least 5%, 10%, 20%, 25%, 30%, 35%, 40%, 45%, 50%, 55%, 60%, 65%, 70%, 75%, 80%, 85%, 90%, 95%, or 100% of internucleoside linkages in the isotopically enriched SMAD7 antisense oligonucleotide are phosphorothioate linkages. In some embodiments, all internucleoside linkages are phosphorothioate linkages.

In some embodiments, the isotopically enriched SMAD7 antisense oligonucleotides include at least one, unnatural nucleoside, e.g., 5-methyl-2′-deoxycytidine-5′-monophosphate and 5-methyl-2′-deoxycytidine-5′-monophosphorothioate. In some embodiments, the isotopically enriched SMAD7 antisense oligonucleotides include 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidines. In some embodiments, at least 5%, 10%, 20%, 25%, 30%, 35%, 40%, 45%, 50%, 55%, 60%, 65%, 70%, 75%, 80%, 85%, 90%, 95%, or 100% of nucleotides in the isotopically enriched SMAD7 antisense oligonucleotide include deoxycytidine and/or 5-methyl-2′-deoxycytidine. In some embodiments, the isotopically enriched SMAD7 antisense oligonucleotides include at least 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidine. In some embodiments, the isotopically enriched SMAD7 antisense oligonucleotides include one or more deoxycytidines and no 5-methyl 2′-deoxycytidine. In some embodiments, the isotopically enriched SMAD7 antisense oligonucleotides include one or more 5-methyl 2′-deoxycytidine and no deoxycytidine.

In some embodiments, the isotopically enriched SMAD7 antisense oligonucleotides include the nucleic acid sequence: 5′-GTXGCCCCTTCTCCCXGCAG-3′ (SEQ ID NO: 8), wherein X is 5-methyl-2′-deoxycytidine and wherein all internucleotide linkages are phosphorothioate linkages.

In some embodiments, the isotopically enriched SMAD7 antisense oligonucleotides include the nucleic acid sequence 5′-GTXGCCCCTTCTCCCXGCAGC-3′ (SEQ ID NO: 9), wherein X is 5-methyl-2′-deoxycytidine and wherein all internucleotide linkages are phosphorothioate linkages.

In some embodiments, the isotopically enriched SMAD7 antisense oligonucleotides include methylphosphonate linkages that are be placed at the 5′- and/or 3′-ends of the isotopically enriched SMAD7 antisense oligonucleotides.

In some embodiments, the isotopically enriched SMAD7 antisense oligonucleotides include pharmaceutically acceptable salts or solvates. In some embodiments, the solvates are hydrates. In some embodiments, the isotopically enriched SMAD7 antisense oligonucleotides are a sodium salt of the isotopically enriched SMAD7 antisense oligonucleotide including the nuclei acid sequence of SEQ ID NO: 7, that optionally can include 1 to 20 O,O-linked phosphorothioate internucleotide linkages. Contemplated salts of isotopically enriched SMAD7 antisense oligonucleotides include those that are fully neutralized, e.g., each phosphorothioate linkage is associated with an ion such as Na⁺. In some embodiments the salts of the isotopically enriched SMAD7 antisense oligonucleotides are only partially neutralized, e.g., less than all phosphorotioate linkages are associated with an ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized).

Exemplary SMAD7 antisense oligonucleotides that can be isotopically enriched, are be described in U.S. Pat. Nos. 6,159,697, 7,807,818, and 8,648,186 and in International Patent Application Publication WO 2010/054826, each of which is incorporated herein by reference.

In some embodiment, the isotopically enriched SMAD7 antisense oligonucleotide includes a SMAD7 antisense oligonucleotide of Formula (I). The following structure of Formula (I) is drawn over four pages:

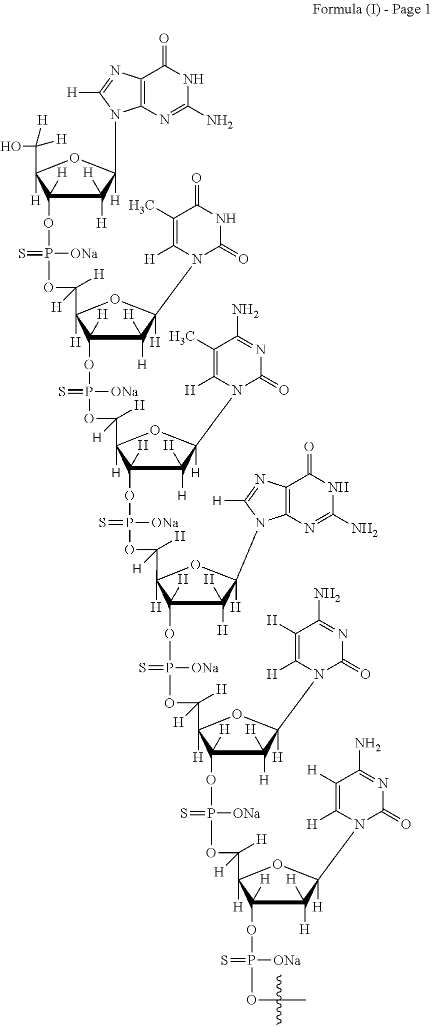

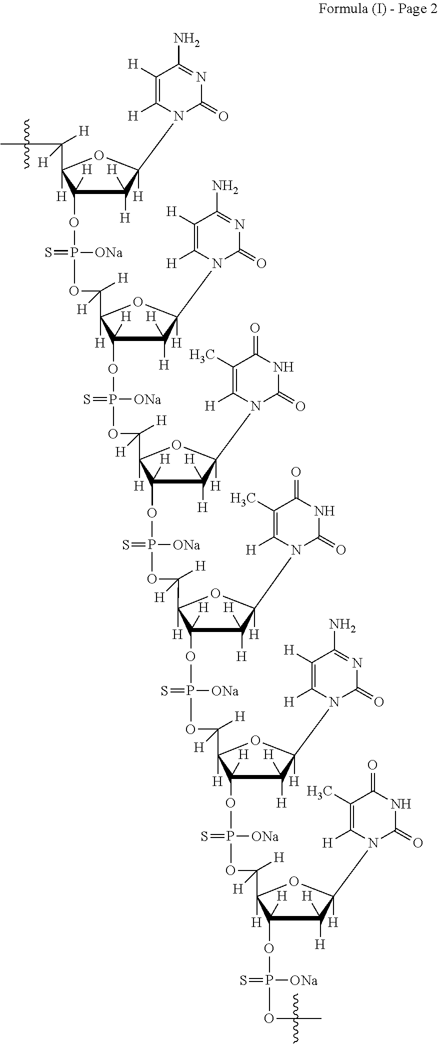

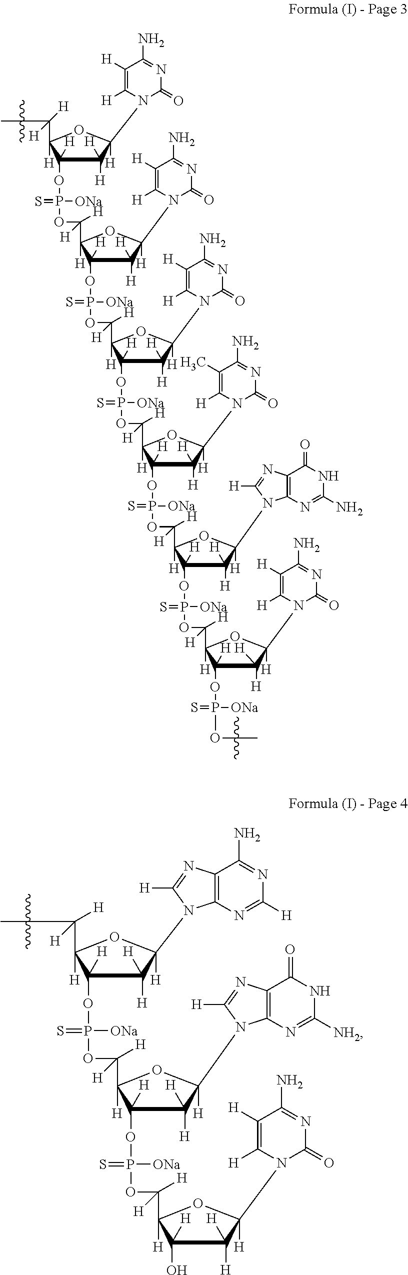

or a pharmaceutically acceptable salt or solvate thereof, wherein one or more H are replaced by D.

In some embodiments, the 2′-deoxyribonucleotides in the isotopically enriched SMAD7 antisense oligonucleotides provided herein are replaced by corresponding ribonucleotides.

### Deuteration

The deuterated anti-SMAD7 agents provided herein, including deuterated SMAD7 antisense oligonucleotides, can be synthesized using methods known to those of ordinary skill in the art. For example, SMAD7 antisense oligonucleotides can be prepared using standard oligonucleotide synthesis methods known to those skilled in the art. Deuterated SMAD7 antisense oligonucleotides can prepared, e.g., by exchanging one or more H in a anti-SMAD7 agent of interest with D, or by synthesizing the anti-SMAD7 agent of interest with D-enriched starting material. See, e.g., U.S. Patent Publication Nos. 2014/0039175 (see especially “Description of the Prior Art,” paragraphs [0013]-[0027]) and 2014/0142141; Maeda et al. (2009) Tetrahedron Letters 16 (19-20), 1643-1646; Sajiki et al. (2003) Nucleic Acids Research Supplement No. 3, 55-56. Deuterated starting materials for the synthesis of deuterated SMAD7 antisense oligonucleotides are commercially available, e.g., from Sigma-Aldrich (Saint Louis, Mo.), Glen Research (Sterling, Va.) and Omicron Biochemicals, Inc. (South Bend, Ind.).

One of ordinary skill in the art understands that in all compounds having a hydrogen (H), including nucleic acids, the hydrogen atom represents a mixture of hydrogen and deuterium (D). The natural abundance of D is about 0.015% of H present in a compound or composition. It will therefore be recognized by one of ordinary skill in the art that a compound or composition is deuterated if the level of D in the compound or composition exceeds the natural level of about 0.015%. For example, a compound is D-enriched or deuterated if at least 0.02%, at least 0.05%, at least 0.1%, at least 0.2%, at least 0.5%, at least 1.0%, at least 2.%, or a higher percentage of H in the compound or composition, up to and including 100% of H, are replaced with D.

A person of ordinary skill in the art will further appreciate that H-atoms present in SMAD7 antisense oligonucleotides can vary in their capacity for exchange with D. Many factors are understood to affect the exchange, including the mechanism of the chemical exchange reaction (e.g., acid-base catalysis), reaction conditions, such as pH and temperature, and considerations relating to the molecular structure of SMAD7 antisense oligonucleotides, e.g., an H-atoms involvement in hydrogen bonding interactions. Generally, more labile protons are more likely to exchange with deuterium.

In some embodiments, the isotopically enriched SMAD7 antisense oligonucleotides provided herein are deuterated SMAD7 antisense oligonucleotides.

The deuterated SMAD7 antisense oligonucleotides have a plurality of H and can have any number of H replaced with D. In some embodiments, only a single H is replaced with D. In some embodiments, all H are replaced with D. In some embodiments, at least 1%, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of H are replaced with deuterium.

The H replaced by D can be enriched in D to any degree that is higher than the natural occurrence of D. In some embodiments, the H replaced with D is enriched in D to more than 0.03% D, more than 0.1% D, more than 0.3% D, more than 1% D, more than 3% D, more than 10% D, more than 15% D, more than 20% D, more than 25% D, more than 25% D, more than 30% D, more than 35% D, more than 40% D, more than 45% D, more than 50% D, more than 55% D, more than 60% D, more than 65% D, more than 70% D, more than 75% D, more than 80% D, more than 85% D, more than 90% D, more than 95% D, more than 98% D, more than 99% D, or 100% D. In some embodiments, the H replaced with D is enriched in D to less than 100% D, less than 99% D, less than 98% D, less than 95% D, less than 90% D, less than 85% D, less than 80% D, less than 75% D, less than 70% D, less than 65% D, less than 60% D, less than 55% D, less than 50% D, less than 45% D, less than 40% D, less than 35% D, less than 30% D, less than 25% D, less than 20% D, less than 15% D, less than 10% D, less than 5% D, less than 3% D, less than 1% D, less than 0.3% D, less than 0.1% D or less than 0.03% D.

Deuterium can be distributed statistically across the deuterated SMAD7 antisense oligonucleotides (e.g., by deuterating SMAD7 antisense oligonucleotides with D₂O). In some embodiments, a larger fraction of relatively labile H (e.g., H attached to a heteroatom) in the deuterated SMAD7 antisense oligonucleotides are replaced, compared to the smaller fraction of less labile H (e.g., H attached to deoxyribose) replaced. In some embodiments, a smaller fraction of relatively labile H (e.g., H attached to a heteroatom) in the deuterated SMAD7 antisense oligonucleotides are replaced, compared to the larger fraction of less labile H (e.g., H attached to deoxyribose) replaced.

In some embodiments, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of nucleotides in a deuterated SMAD7 antisense oligonucleotides are partially deuterated within a preparation of the deuterated SMAD7 antisense oligonucleotides (e.g., H replaced with D in at least 1%, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the deuterated SMAD7 antisense oligonucleotides in the preparation).

In some embodiments, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of nucleotides in the deuterated SMAD7 antisense oligonucleotides are fully deuterated within a preparation of the deuterated SMAD7 antisense oligonucleotide (H replaced with D in essentially 100% of the isotopically enriched SMAD7 antisense oligonucleotides in the preparation).

In some embodiments, the deuterated SMAD7 antisense oligonucleotide includes a SMAD7 antisense oligonucleotide of Formula (II). The following structure of Formula (II) is drawn over four pages:

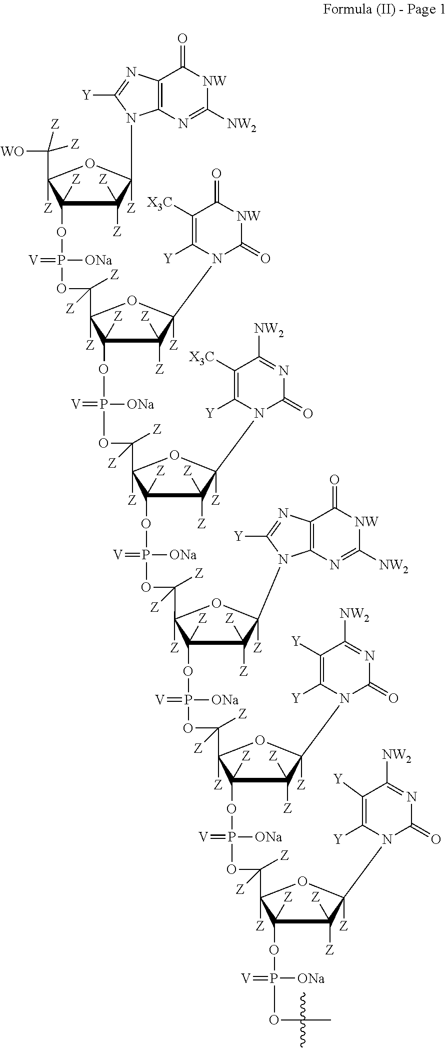

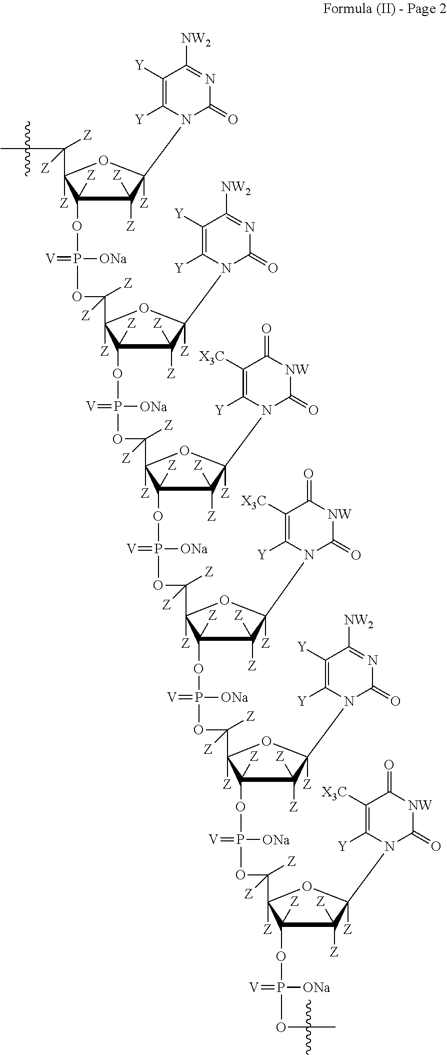

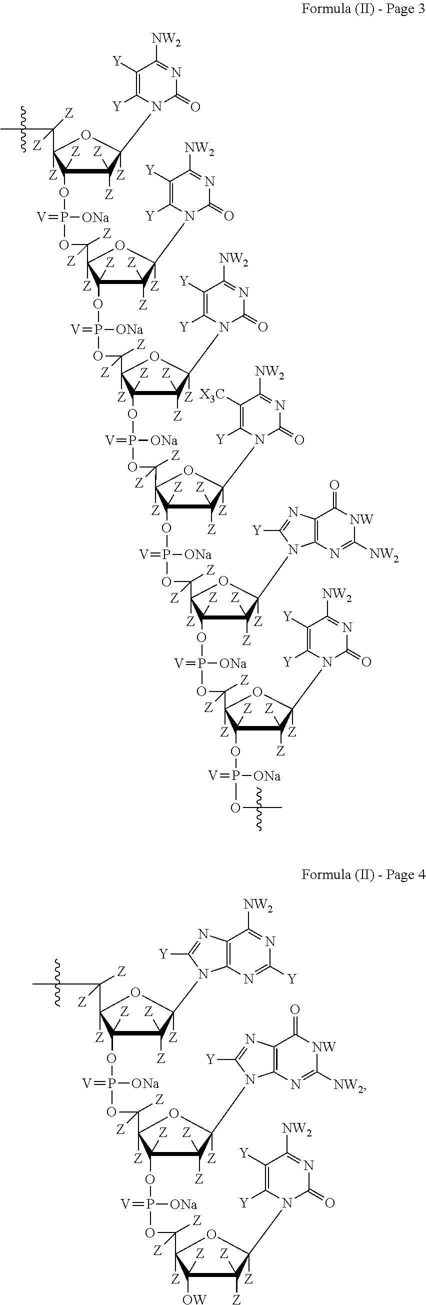

or a pharmaceutically acceptable salt or solvate thereof, wherein each V individually is oxygen or sulfur and W, X, Y, and Z each individually are H or D.

In some embodiments, V is oxygen. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or all 20 V of Formula (II) are oxygen.

In some embodiments, V is sulfur. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or all 20 V of Formula (II) are sulfur.

In some embodiments, more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, or more than 97% of W in Formula (II) are D. In some embodiments 100% of W in Formula (II) are D.

In some embodiments, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of X in Formula (II) are D.

In some embodiments, more than 1%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of Y in Formula (II) are D.

In some embodiments, more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 97%, more than 98%, or more than 99% of Z in Formula (II) are D.

In some embodiments, about the same fraction of W, X, Y and Z in Formula (II) are D. For example, about 50% of W, about 50% of X, about 50% of Y and about 50% of Z are D.

In some embodiments, the fractions of W, X, Y and Z in Formula (II) that are D are different. For example, about 10% of W are D, about 20% of X are D, about 80% of Y are D and about 60% of Z are D.

In some embodiments, the relative sizes of the fractions of W, X, Y and Z of Formula (II) that are D can have any one relationship shown in Table 1.

In some embodiments, the degree of deuteration of any one deuterated W of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%. In some embodiments, the degree of deuteration of any one deuterated W of Formula (II) is 100%.

In some embodiments, the degree of deuteration of different deuterated W of Formula (II) is about the same. In some embodiments, the degree of deuteration of different deuterated W of Formula (II) is different.

In some embodiments, the degree of deuteration of only one deuterated X of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%. In some embodiments, the degree of deuteration of any one deuterated X of Formula (II) is 100%.

In some embodiments, the degree of deuteration of different deuterated X of Formula (II) is about the same. In some embodiments, the degree of deuteration of different deuterated X of Formula (II) is different.

In some embodiments, the degree of deuteration of only one deuterated Y of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%. In some embodiments, the degree of deuteration of any one deuterated Y of Formula (II) is 100%.

In some embodiments, the degree of deuteration of different deuterated X of Formula (II) is about the same. In some embodiments, the degree of deuteration of different deuterated X of Formula (II) is different.

In some embodiments, the degree of deuteration of only one deuterated Z of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%. In some embodiments, the degree of deuteration of any one deuterated Z of Formula (II) is 100%.

In some embodiments, the degree of deuteration of different deuterated Z of Formula (II) is about the same. In some embodiments, the degree of deuteration of different deuterated Z of Formula (II) is different.

In some embodiments, the average, median, or mean degree of deuteration for the deuterated W, X, Y and Z in Formula (II) can be about the same. For example, deuterated W, X, Y and Z of Formula (II) can, on average, be 50% deuterated.

In some embodiments, the average, median or mean degree of deuteration can differ for deuterated W, X, Y and Z of Formula (II). For example, deuterated W are, on average, about 50% deuterated, deuterated X are, on average, about 10% deuterated, deuterated Y are, on average, about 60% deuterated and deuterated Z are, on average, about 90% deuterated.

In some embodiments, the relative average, median or mean degrees of deuteration of deuterated W, X, Y, and Z of Formula (II) can have any one relationship shown in Table 1.

In some embodiments, a larger fraction of Z in Formula (II) is D than of Y, X or W.

TABLE 1
Relative number of W, X, Y and Z of Formula (II) that are D
(e.g., in percent) or relative level of deuteration of W, X, Y
and Z (e.g., in D/H-ratios)

| | |
|---|---|
| 1 | W > or = X > or = Y > or = Z |
| 2 | W > or = X > or = Z > or = Y |
| 3 | W > or = Y > or = X > or = Z |
| 4 | W > or = Z > or = X > or = Y |
| 5 | W > or = Y > or = Z > or = X |
| 6 | W > or = Z > or = Y > or = X |
| 7 | X > or = W > or = Y > or = Z |
| 8 | X > or = W > or = Z > or = Y |
| 9 | X > or = Y > or = W > or = Z |
| 10 | X > or = Z > or = W > or = Y |
| 11 | X > or = Y > or = Z > or = W |
| 12 | X > or = Z > or = Y > or = W |
| 13 | Y > or = W > or = X > or = Z |
| 14 | Y > or = W > or = Z > or = X |
| 15 | Y > or = X > or = W > or = Z |
| 16 | Y > or = Z > or = W > or = X |
| 17 | Y > or = X > or = Z > or = W |
| 18 | Y > or = Z > or = X > or = W |
| 19 | Z > or = W > or = X > or = Y |
| 20 | Z > or = W > or = Y > or = X |
| 21 | Z > or = X > or = W > or = Y |
| 22 | Z > or = Y > or = W > or = X |
| 23 | Z > or = X > or = Y > or = W |
| 24 | Z > or = Y > or = X > or = W |

Deuterium can be introduced into SMAD7 antisense oligonucleotides in a site-specific manner, e.g., by synthesizing the SMAD7 antisense oligonucleotides of interest with deuterated starting materials, such as deuterated nucleotides (e.g., [5′,5″-²H₂]adenosine 5′-monophosphate) or deuterated deoxysugars (e.g., 2-deoxy-D-[1-²H]glucose).

In some embodiments, site specific deuteration can result in the introduction of one or more chiral centers in the deuterated SMAD7 antisense oligonucleotide, e.g., at C2′ or C5′ of a D-ribose or D-deoxyribose in a deuterated SMAD7 antisense oligonucleotide. In some embodiments, more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 75%, more than 80%, more than 85%, more than 90% or more than 95% or C2′ and/or C5′ of a D-ribose or D-deoxyribose in a deuterated SMAD7 antisense oligonucleotide are chiral centers.

In some embodiments, the deuterated SMAD7 antisense oligonucleotides include racemic mixtures. In some embodiments, the SMAD7 antisense oligonucleotides include an enantiomeric excess. In some embodiments, the SMAD7 antisense oligonucleotides include a mixture of diastereomers.

In some embodiments, essentially all D in the deuterated SMAD7 antisense oligonucleotides are present in a nucleobase (e.g., a cytosine or guanine). In some embodiments, essentially all D in the SMAD7 antisense oligonucleotides are present a sugar moiety, e.g., in a ribose or doxyribose.

In some embodiments, the deuterated SMAD7 antisense oligonucleotide includes a SMAD7 antisense oligonucleotide of Formula (III). The following structure of Formula (III) is drawn over four pages:

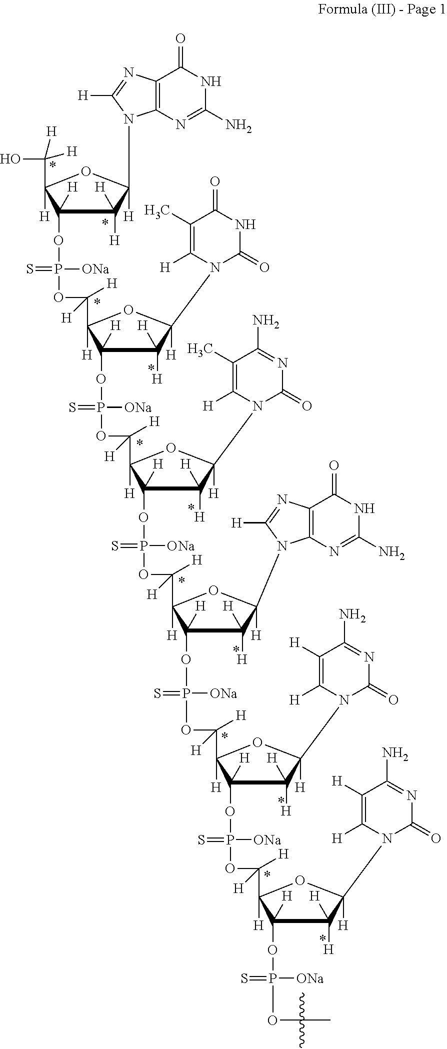

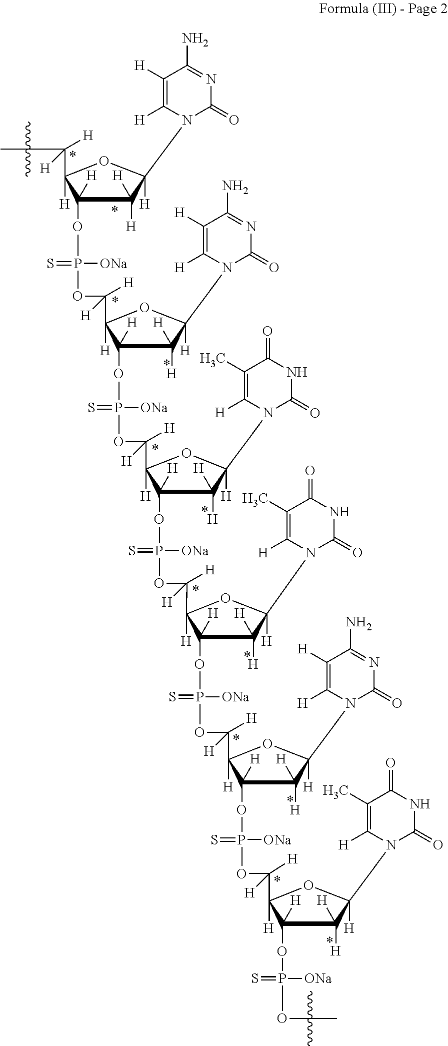

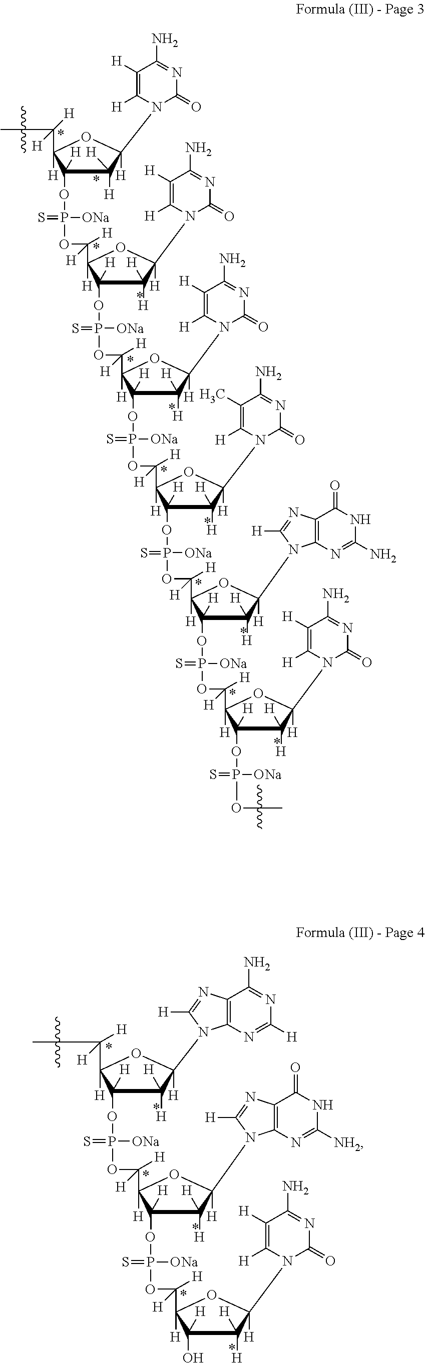

or a pharmaceutically acceptable salt or solvate thereof, wherein one or more H are replaced by D.

In some embodiments, essentially all D in the deuterated SMAD7 antisense oligonucleotides are present in one or more nucleotides of interest. In some embodiments, essentially all D in the deuterated SMAD7 antisense oligonucleotides are present in one or more nucleobases of interest (e.g., adenine, guanine, cytosine, thymine, uracil). In some embodiments, essentially all D in the deuterated SMAD7 antisense oligonucleotides are present in one or more sugar moieties of interest (e.g., ribose or doxyribose moieties) in the deuterated SMAD7 antisense oligonucleotide.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of nucleotides in the deuterated SMAD7 antisense oligonucleotides include one or more D. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 nucleotides in the deuterated SMAD7 antisense oligonucleotides include one or more D. In some embodiments, the one or more nucleotides each include at least 1, at least 2, at least 3, at least 4, at least 5, at least 6, at least 7, at least 8, at least 9, at least 10, at least 11 or at least 12 D. Different deuterated nucleotides can have the same number of D or different numbers of D.

In some embodiments, one or more nucleobases of interest in the deuterated SMAD7 antisense oligonucleotides are deuterated. In some embodiments, each of the one or more nucleobases of interest comprises at least 1, at least 2, at least 3, at least 4, or at least 5 D. The one or more nucleobases of interest can include the same number of D or different numbers of D.

In some embodiments, the one or more deuterated nucleobases of interest in the deuterated SMAD7 antisense oligonucleotide include a purine. In some embodiments, the one or more deuterated nucleobase of interest in the deuterated SMAD7 antisense oligonucleotide include an adenine. In some embodiments, the one or more deuterated nucleobase of interest in the deuterated SMAD7 antisense oligonucleotide include a guanine.

In some embodiments, the one or more deuterated nucleobase of interest in the deuterated SMAD7 antisense oligonucleotide include a pyrimidine. In some embodiments, the one or more deuterated nucleobase of interest in the deuterated SMAD7 antisense oligonucleotide include a cytosine. In some embodiments, the one or more deuterated nucleobase of interest in the deuterated SMAD7 antisense oligonucleotide include a thymine. In some embodiments, the one or more deuterated nucleobase of interest in the deuterated SMAD7 antisense oligonucleotide include a uracil.

Any hydrogen in a nuclebase of interest can be deuterated individually or in combination with any other hydrogen in the nucleobase of interest. In some embodiments, the hydrogens bound to C2, C8, or the amino group of an adenine are deuterated individually or in any combination. In some embodiments, the hydrogens bound to C8 or the amino group of a guanine are deuterated individually or in any combination. In some embodiments, the hydrogens bound to the C6, N3 or the methyl-group in thymine, or the C5, C6 and N3 of a thymine are deuterated individually or in any combination. In some embodiments, the hydrogens bound to the C5, C6 or the amino group of cytosine are deuterated individually or in any combination.

In some embodiments, one or more sugar moieties of interest in the deuterated SMAD7 antisense oligonucleotides are deuterated. In some embodiments, the one or more sugar moieties of interest include a deoxyribose. In some embodiments, the one or more sugar moieties include a ribose. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of sugar moieties in the deuterated SMAD7 antisense oligonucleotides include one or more D. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 sugar moieties in the deuterated SMAD7 antisense oligonucleotides include one or more D. In some embodiments, the one or more sugar moieties each include at least 1, at least 2, at least 3, at least 4, at least 5, at least 6, at least 7, at least 8, at least 9, at least 10, at least 11 or at least 12 D. Different sugar moieties can include the same number of D or different numbers of D.

### Other Isotopologues

In some embodiments, the isotopically enriched SMAD7 antisense oligonucleotides can be enriched in isotopologues, such as tritium (T), ¹³C, ³³S, ³⁴S, ³⁶S, ¹⁵N and ¹⁷O. In some embodiments, the isotopically enriched SMAD7 antisense oligonucleotides can be fluorinated.

In some embodiments, the isotopically enriched SMAD7 antisense oligonucleotides include include a plurality of hydrogens (H), wherein one or more hydrogens of the plurality of hydrogens are replaced by tritium (T). In some embodiments, one or more nucleotides of the isotopically enriched SMAD7 antisense oligonucleotides can include one or more H that are replaced by T. In some embodiments, the one or more tritiated nucleotides include one or more T in a sugar moiety (e.g., a ribose or deoxyribose) or a nucleobase (e.g., a purine or pyrimidine base), or any combination thereof. In some embodiments any one of W, X, Y or Z of Formula (II) or any combination thereof can be tritiated. Any H replaced with a T can be partially or fully tritiated.

In some embodiments, the isotopically enriched SMAD7 antisense oligonucleotides include include a plurality of hydrogens (H), wherein one or more hydrogens of the plurality of hydrogens are replaced by a fluor (F). In some embodiments, one or more nucleotides of the isotopically enriched SMAD7 antisense oligonucleotides can include one or more H that are replaced by F. In some embodiments, the one or more fluorinated nucleotides include one or more F in a sugar moiety (e.g., a ribose or deoxyribose) or a nucleobase (e.g., a purine or pyrimidine base), or any combination thereof. In some embodiments any one of W, X, Y or Z of Formula (II) or any combination thereof can be fluorinated.

In some embodiments, the isotopically enriched SMAD7 antisense oligonucleotides include include a plurality of carbons (C), wherein one or more carbon of the plurality of carbons are replaced by ¹³C. In some embodiments, one or more nucleotides of the isotopically enriched SMAD7 antisense oligonucleotides can include one or more C that are replaced by ¹³C. In some embodiments, the one or more ¹³C-containing nucleotides include one or more ¹³C in a sugar moiety (e.g., a ribose or deoxyribose) or a nucleobase (e.g., a purine or pyrimidine base), or any combination thereof. Any C replaced with a ¹³C can be partially or fully replaced.

In some embodiments, the isotopically enriched SMAD7 antisense oligonucleotides include include a plurality of oxygens (O), wherein one or more oxygen of the plurality of oxygens are replaced by ³³S, ³⁴S, ³⁶S, or ¹⁷O. In some embodiments, one or more nucleotides of the isotopically enriched SMAD7 antisense oligonucleotides can include one or more O that are replaced by ³³S, ³⁴S, ³⁶S, ¹⁵N or ¹⁷O. In some embodiments, the one or more ³³S, ³⁴S, ³⁶S or ¹⁷O-containing nucleotides include one or more ³³S, ³⁴S, ³⁶S, ¹⁵N or ¹⁷O in a sugar moiety (e.g., a ribose or deoxyribose) or a nucleobase (e.g., a purine or pyrimidine base), or any combination thereof. Any O replaced with a ³³S, ³⁴S, ³⁶S or ¹⁷O can be partially or fully replaced. One or more V in Formula (II) can be ³³S, ³⁴S, ³⁶S or ¹⁷O.

In some embodiments, the isotopically enriched SMAD7 antisense oligonucleotides include include a plurality of nitrogens (N), wherein one or more nitrogens of the plurality of nitrogens are replaced by ¹⁵N. In some embodiments, one or more nucleotides of the isotopically enriched SMAD7 antisense oligonucleotides can include one or more N that are replaced by ¹⁵N. In some embodiments, the one or more ¹⁵N-containing nucleotides include one or more ¹⁵N in a sugar moiety (e.g., an amino-sugar) or a nucleobase (e.g., a purine or pyrimidine base), or any combination thereof. Any N replaced with a ¹⁵N can be partially or fully replaced.

### Pharmaceutical Compositions

In another aspect, provided herein is a pharmaceutical composition including an isotopically enriched SMAD7 antisense oligonucleotide provided herein and a pharmaceutically acceptable adjuvant and/or excipient. In some embodiments, the pharmaceutical composition is an oral pharmaceutical composition. In some embodiments, the pharmaceutical composition includes an enteric coating to topically deliver the modified SMAD7 antisense oligonucleotide to the terminal ileum and/or right colon of an IBD patient.

Contemplated modified SMAD7 antisense oligonucleotides include isotopically enriched oligonucleotides that act against SMAD7 and can be administered orally. Disclosed therapies can, when administered orally to a subject suffering from IBD, deliver an effective amount of an isotopically enriched antisense oligonucleotide to the intestinal system of a patient, e.g. deliver an effective amount of an isotopically enriched antisense oligonucleotide to the terminal ileum and/or right colon of a patient.

In some embodiments of the invention, the anti-SMAD7 therapy (e.g., a therapy comprising an isotopically enriched SMAD7 antisense oligonucleotide) can be suitable for oral delivery of an antisense oligonucleotide, e.g., tablets, that include an enteric coating, e.g., a gastro-resistant coating, such that the compositions can deliver the antisense compound to, e.g., the terminal ileum and right colon of a patient. For example, such administration can result in a topical effect, substantially topically applying the antisense compound directly to an affected portion of the intestine of a subject. Such administration, can, in some embodiments, substantially avoid unwanted systemic absorption of the antisense compound.

For example, a tablet for oral administration can comprise granules (e.g., is at least partially formed from granules) that include a disclosed isotopically enriched SMAD7 antisense oligonucleotide and pharmaceutically acceptable excipients. Such a tablet can be coated with an enteric coating. Contemplated tablets can include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a contemplated isotopically enriched SMAD7 antisense oligonucleotide or a pharmaceutically acceptable salt and a pharmaceutically acceptable filler. For example, an isotopically enriched SMAD7 antisense oligonucleotide including the nucleic acid sequence of SEQ ID NO: 9 and a filler can be blended together, with optionally other excipients, and formed into granules. In some embodiments, the intragranular phase can be formed using wet granulation, e.g. a liquid (e.g., water) is added to the blended antisense compound and filler, and then the combination is dried, milled and/or sieved to produce granules. One of skill in the art would understand that other processes can be used to achieve an intragranular phase.

In some embodiments, contemplated formulations include an extra-granular phase, which can include one or more pharmaceutically acceptable excipients, and which can be blended with the intragranular phase to form a disclosed formulation.

An anti-SMAD7 therapy formulation can include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, an anti-SMAD7 therapy formulation can include an intragranular phase and/or an extragranular phase that includes a binder, which can generally function to hold the ingredients of the pharmaceutical formulation together. Exemplary binders include, for example, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others, including combinations thereof.

Contemplated anti-SMAD7 therapy formulations, e.g., that include an intragranular phase and/or an extragranular phase, can include a disintegrant, such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase can include a disintegrant.

In some embodiments, a contemplated anti-SMAD7 therapy formulation includes an intra-granular phase comprising a disclosed antisense compound and excipients chosen from: mannitol, microcrystalline cellulose, hydroxypropylmethyl cellulose, and sodium starch glycolate, or combinations thereof, and an extra-granular phase comprising one or more of: microcrystalline cellulose, sodium starch glycolate, and magnesium stearate, or mixtures thereof.

In some embodiments, a contemplated anti-SMAD7 therapy formulation can include a lubricant, e.g., an extra-granular phase can contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings can include a polymer that disintegrates a different rates according to pH. Enteric coatings can include, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In some embodiments, the enteric coating includes an anionic, cationic, or neutral copolymer based on methacrylic acid, methacrylic/acrylic esters or their derivatives. In some embodiments, the enteric coating includes an ethylacrylate-methacrylic acid copolymer. Commercially available enteric coatings include Opadry® AMB, Acryl-EZE®, Eudragit® grades. In some embodiments, the enteric coating makes up about 5% to about 10%, about 5% to about 20%, about 8 to about 15%, about 8% to about 18%, about 10% to about 12%, or about 12% to about 16%, of a contemplated tablet by weight.

For example, an anti-SMAD7 therapy in the form of a tablet is provided that comprises or consists essentially of about 0.5% to about 70%, e.g., about 0.5% to about 10%, or about 1% to about 20%, by weight of an isotopically enriched SMAD7 antisense oligonucleotide or a pharmaceutically acceptable salt thereof. Such a tablet can include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a contemplated tablet can comprise an intragranular phase that includes about 30% to about 60%, e.g., about 45% to about 65% by weight, or alternatively, about 5 to about 10% by weight of a modified SMAD7 antisense oligonucleotide including the nucleic acid sequence of SEQ ID NO: 9, about 30% to about 50%, or alternatively, about 5% to about 15% by weight mannitol, about 5% to about 15% microcrystalline cellulose, about 0% to about 4%, or about 1% to about 7% hydroxypropylmethyl cellulose, and about 0% to about 4%, e.g. about 2% to about 4% sodium starch glycolate by weight.

Exemplary anti-SMAD7 therapy formulations include dosage forms that include or consist essentially of about 10 mg to about 500 mg of an isotopically enriched SMAD7 antisense oligonucleotide including the nucleic acid sequence of SEQ ID NO:9, for example, tablets that include about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 150 mg, about 200 mg, or about 250 mg of an isotipically enriched SMAD7 antisense oligonucleotide including the nucleic acid sequence of SEQ ID NO: 9 are contemplated herein. In one embodiment, the anti-SMAD7 therapy can be a tablet for oral use comprising: about 0.5% to about 10% by weight of an antisense oligonucleotide represented by SEQ ID NO: 9 or a pharmaceutically acceptable salt thereof; about 30% to about 50% by weight mannitol; and about 10% to about 30% by weight microcrystalline cellulose.

In an exemplary embodiment of the invention, a pharmaceutically acceptable tablet for oral administration is provided that includes an intra-granular phase that can comprise about 50% by weight of an isotopically enriched SMAD7 antisense oligonucleotide including the nucleic acid sequence of SEQ ID NO: 9 (or salt thereof), about 11.5% by weight mannitol, about 10% by weight microcrystalline cellulose, about 3% by weight hydroxypropylmethyl cellulose, and about 2.5% by weight sodium starch glycolate; and an extra-granular phase that can comprise about 20% by weight microcrystalline cellulose, about 2.5% by weight sodium starch glycolate, and about 0.5% by weight magnesium stearate. The tablet can also include an enteric coating.

In another exemplary embodiment, a pharmaceutically acceptable tablet for oral administration is provided that includes or consists essentially of: an intra-granular phase that can comprise or consist essentially of about 5% to about 10%, e.g., about 8% by weight of an isotopically enriched SMAD7 antisense oligonucleotide including the nucleic acid sequence of SEQ ID NO: 9 (e.g., wherein the internucleotide linkages are each O,O-linked phophorothioates, and/or salt thereof, e.g., a sodium salt), about 40% by weight mannitol, about 8% by weight microcrystalline cellulose, about 5% by weight hydroxypropylmethyl cellulose, and about 2% by weight sodium starch glycolate; and an extra-granular phase that can comprise about 17% by weight microcrystalline cellulose, about 2% by weight sodium starch glycolate, and about 0.4% by weight magnesium stearate.

Contemplated tablets can also include an enteric coating, e.g., a disclosed tablet can include about 13%, about 14%, about 15%, about 16%, about 17% by weight of an enteric coating, e.g., ethylacrylate-methacrylic acid copolymers (e.g., AcyrlEZE®).

For example, the anti-SMAD7 therapy can be in the form of a pharmaceutically acceptable tablet for oral use comprising an intra-granular phase and extra-granular phase, wherein for example, the intra-granular phase comprises about 5% to about 10%, by weight (for example about 8% by weight) of a modified SMAD7 antisense oligonucleotide including the nucleic acid sequence of SEQ ID NO: 9 or a pharmaceutically acceptable salt thereof, about 40% by weight mannitol, about 8% by weight microcrystalline cellulose, about 5% by weight hydroxypropylmethyl cellulose, and about 2% by weight sodium starch glycolate, and, e.g., the extra-granular phase comprises about 17% by weight microcrystalline cellulose, about 2% by weight sodium starch glycolate, and about 0.4% by weight magnesium stearate, where the tablet can further comprise an enteric coating.

Contemplated formulations, e.g., tablets, in some embodiments, when orally administered to the patient can result in minimal plasma concentration of the oligonucleotide in the patient. In another embodiment, contemplated formulations, when orally administered to a patient, topically deliver to the terminal ileum and/or right colon of a patient, e.g., to an affected or diseased intestinal site of a patient.

### Uses

In another aspect, provided herein is a method of treating inflammatory bowel disease (IBD), comprising administering to a patient in need thereof an effective amount of an isotopically enriched SMAD7 antisense oligonucleotide provided herein, wherein the isotopically enriched SMAD7 antisense oligonucleotide is effective to treat or manage IBD.

In some embodiments, the IBD is Crohn's disease (CD).

In some embodiments, the IBD is ulcerative colitis (UC).

In some embodiments, the isotopically enriched SMAD7 antisense oligonucleotide is administered orally.

“Inflammatory bowel disease” or “IBD”, as used herein, can refer to a number of chronic inflammatory diseases including Crohn's disease (CD), gastroduodenal Crohn's disease, Crohn's (granulomatous) colitis, ulcerative colitis (UC), collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis. CD and UC are the two most common forms of IBD. IBD is an autoimmune disease of the digestive system. CD can be localized to any portion of the gastrointestinal tract, including the terminal ileum, and can impact all cell types of the gastrointestinal tract. UC is localized to the colon and rectum, and affects cells of the mucosa only.

Both environmental and genetic factors are believed to play a role in IBD, although the identity of such factors is not well-defined. Environmental components can include alterations in flora of the gut which are affected by exposure to ingested foods and medications.

IBD is associated with symptoms including abdominal pain, vomiting, diarrhea, rectal bleeding, severe cramps, muscle spasms, weight loss, malnutrition, fever, anemia, skin lesions, joint pain, eye inflammation, liver disorders, arthritis, pyoderma gangrenosum, primary sclerosing cholangitis, and non-thyroidal illness syndrome. Children suffering from UC can suffer from growth defects.

Forms of CD include steroid-dependent and steroid-resistant forms of CD, including active CD. Patients with IBD who suffer from a steroid-dependent form of CD are responsive to treatment with steroid therapy, but cannot terminate or curtail steroid therapy without suffering from an increase in occurrence of symptoms associated with CD. Patients with IBD who suffer from a steroid-resistant form of CD are not responsive to treatment with steroid therapy. Steroid therapeutics commonly prescribed and/or administered to patients with IBD include: corticosteroids, for example, prednisone, dexamethasone, hydrocortisone, methylprednisolone, prednisone, and budesonide. A human patient suffering from active CD is a patient actively suffering from symptoms of CD, for example, but not limited to, bloody stool, weight loss, and/or abdominal cramps.

A “subject” or “patient” as described herein, refers to any animal at risk for, suffering from or diagnosed for IBD, including, but not limited to, mammals, primates, and humans. In certain embodiments, the subject may be a non-human mammal such as, e.g., a cat, a dog, or a horse. In a preferred embodiment, the subject is a human subject. A subject may be an individual diagnosed with a high risk of developing IBD, someone who has been diagnosed with IBD, someone who previously suffered from IBD, or an individual evaluated for symptoms or indications of IBD, for example, a high CDAI index score.

“A patient with IBD,” as used herein, refers to a patient suffering from any of the symptoms or manifestations of IBD, a patient who may suffer from any of the symptoms or manifestations of IBD, or any patient who might benefit from a method of the invention for treating or evaluating treatment for IBD. A patient in need may include a patient who is diagnosed with a risk of developing IBD, a patient who has suffered from IBD in the past, or a patient who has previously been treated for IBD.

The terms “treat,” “treatment,” “treating,” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. preventing the disease from increasing in severity or scope; (c) relieving the disease, i.e. causing partial or complete amelioration of the disease; or (d) preventing relapse of the disease, i.e. preventing the disease from returning to an active state following previous successful treatment of symptoms of the disease or treatment of the disease.

The terms “manage,” “management,” “managing,” and the like are used herein to generally mean controlling the severity or manifestation of symptoms of a disease, or the means of treating the disease. Generally, management is used to obtaining a desired pharmacological and/or physiological effect. The effect may be therapeutic in terms of partially or completely curing a disease and/or adverse effect attributed to the disease or ensuring that a particular symptom or manifestation of the disease does not occur or reoccur in a patient or does not rise to an undesirable or intolerable level in a patient. The term “management” as used herein covers any management of a disease in a mammal, particularly a human, and includes: (a) inhibiting the disease, i.e. preventing the disease from increasing in severity or scope; (b) relieving the disease, i.e. causing partial or complete amelioration of the disease; or (c) preventing relapse of the disease, i.e. preventing the disease from returning to an active state following previous successful treatment of symptoms of the disease or treatment of the disease. “Management” as used herein may also be used with reference to administration of a specific treatment for the disease, for example, an isotopically enriched SMAD7 antisense oligonucleotide.

TABLE 2
SEQUENCE LISTING

| ID | SEQUENCE |
|---|---|
| SEQ ID NO: 1 | ATG TTCAGGACCA AACGATCTGC GCTCGTCCGG CGTCTCTGGA |
| | GGAGCCGTGC GCCCGGCGGC GAGGACGAGG AGGAGGGCGC |
| | AGGGGGAGGT GGAGGAGGAG GCGA [symbol] [symbol] |
| | [symbol] GGACA GCCGAGCGCA TGGGGCCGGT GGCGGCGGCC |
| | CGGGCAGGGC TGGATGCTGC CTGGGCAAGG CGGTGCGAGG |
| | TGCCAAAGGT CACCACCATC CCCACCCGCC AGCCGCGGGC |
| | GCCGGCGCGG CCGGGGGCGC CGAGGCGGAT CTGAAGGCGC |
| | TCACGCACTC GGTGCTCAAG AAACTGAAGG AGCGGCAGCT |
| | GGAGCTGCTG CTCCAGGCCG TGGAGTCCCG CGGCGGGACG |
| | CGCACCGCGT GCCTCCTGCT GCCCGGCCGC CTGGACTGCA |
| | GGCTGGGCCC GGGGGCGCCC GCCGGCGCGC AGCCTGCGCA |
| | GCCGCCCTCG TCCTACTCGC TCCCCCTCCT GCTGTGCAAA |
| | GTGTTCAGGT GGCCGGATCT CAGGCATTCC TCGGAAGTCA |
| | AGAGGCTGTG TTGCTGTGAA TCTTACGGGA AGATCAACCC |
| | CGAGCTGGTG TGCTGCAACC CCCATCACCT TAGCCGACTC |
| | TGCGAACTAG AGTCTCCCCC CCCTCCTTAC TCCAGATACC |
| | CGATGGATTT TCTCAAACCA ACTGCAGACT GTCCAGATGC |
| | TGTGCCTTCC TCCGCTGAAA CAGGGGGAAC GAATTATCTG |
| | GCCCCTGGGG GGCTTTCAGA TTCCCAACTT CTTCTGGAGC |
| | CTGGGGATCG GTCACACTGG TGCGTGGTGG CATACTGGGA |
| | GGAGAAGACG AGAGTGGGGA GGCTCTACTG TGTCCAGGAG |
| | CCCTCTCTGG ATATCTTCTA TGATCTACCT CAGGGGAATG |
| | GCTTTTGCCT CGGACAGCTC AATTCGGACA ACAAGAGTCA |
| | GCTGGTGCAG AAGGTGCGGA GCAAAATCGG CTGCGGCATC |
| | CAGCTGACGC GGGAGGTGGA TGGTGTGTGG GTGTACAACC |
| | GCAGCAGTTA CCCCATCTTC ATCAAGTCCG CCACACTGGA |
| | CAACCCGGAC TCCAGGACGC TGTTGGTACA CAAGGTGTTC |
| | CCCGGTTTCT CCATCAAGGC TTTCGACTAC GAGAAGGCGT |
| | ACAGCCTGCA GCGGCCCAAT GACCACGAGT TTATGCAGCA |
| | GCCGTGGACG GGCTTTACCG TGCAGATCAG CTTTGTGAAG |
| | GGCTGGGGCC AGTGCTACAC CCGCCAGTTC ATCAGCAGCT |
| | GCCCGTGCTG GCTAGAGGTC ATCTTCAACA GCCGGTAG |
| SEQ ID NO: 2 | 5′-GTCGCCCCTTCTCCCCGCAG-3′ |
| SEQ ID NO: 3 | 5′-GTCGCCCCTTCTCCCCGCAGC-3′ |
| SEQ ID NO: 4 | 5′-GTXGCCCCTTCTCCCXGCAG-3′, wherein X is 5-methyl 2′- |
| | deoxycytidine |
| SEQ ID NO: 5 | 5′-GTXGCCCCTTCTCCCXGCAGC-3′, wherein X is 5-methyl 2′- |
| | deoxycytidine |
| SEQ ID NO: 6 | 5′-GTXYCCCCTTCTCCCXYCAG-3′, whereby X is a nucleotide |
| | including a nitrogenous base selected from the group consisting of cytosine |
| | and 5-methylcytosine nucleoside or a 2′-O-methylcytosine nucleoside, and |
| | wherein Y is a nucleotide, selected from the group consisting of guanine |
| | and 5-methylguanine or a 2′-O-methylguanine nucleoside, optionally |
| | provided that at least one of the nucleotides X or Y comprises a methylated |
| | nitrogenous base |
| SEQ ID NO: 7 | 5′-GTC* GCC CCT TCT CCC C*GC AGC-3′), whereby C* represents 5- |
| | methyl-2′-deoxycytidine. |
| | In some embodiments, at least one of the internucleotide linkages of the |
| | isotopically enriched SMAD7 antisense oligonucleotide is an O,O-linked |
| | phosphorothioate, e.g., each of the 20 internucleotide linkages of SEQ ID |
| | NO: 5 can be an O,O-linked phosphorothioate. |
| | In some embodiments, the isotopically enriched SMAD7 antisense |
| | oligonucleotide is an antisense oligonucleotide comprising a nucleotide |
| | sequence of SEQ ID NO: 5, wherein each of the 20 internucleotide |
| | linkages is an O,O-linked phosphorothioate linkage |
| SEQ ID NO: 8 | 5′-GTXGCCCCTTCTCCCXGCAG-3′, wherein X is a 5-methyl-2′- |
| | deoxycytidine and wherein all internucleotide linkages are |
| | phosphorothioate linkages. |
| SEQ ID NO: 9 | 5′-GTXGCCCCTTCTCCCXGCAGC-3′, wherein X is comprising 5- |
| | methyl-2′-deoxycytidine and wherein all internucleotide linkages are |
| | phosphorothioate linkages. |

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1. A deuterated SMAD7 antisense oligonucleotide, comprising a plurality of hydrogens (H), wherein one or more hydrogens of the plurality of hydrogens are replaced by deuterium (D).

2. The deuterated SMAD7 antisense oligonucleotide of claim 1, wherein the one or more hydrogen replaced by deuterium is enriched in deuterium to more than 0.02%, more than 0.03%, more than 0.1%, more than 0.3%, more than 1%, more than 3%, more than 10%, more than 15%, more than 20%, more than 25%, more 30%, more than 35%, more than 40%, more than 45%, more than 50%, more than 55%, more than 60%, more than 65%, more than 70%, more than 75%, more than 80%, more than 85%, more than 90%, more than 95%, more than 98% or more than 99%.

3. The deuterated SMAD7 antisense oligonucleotide of claim 1, wherein at least 1%, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of hydrogens of the plurality of hydrogens are replaced with deuterium.

4. The deuterated SMAD7 antisense oligonucleotide of claim 1, further comprising a plurality of nucleotides, wherein at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of nucleotides of the plurality of nucleotides are partially or fully deuterated.

5. The deuterated SMAD7 antisense oligonucleotide of claim 1, further comprising a plurality of nucleobases, wherein one or more nucleobases of the plurality of nucleobases are deuterated.

6. The deuterated SMAD7 antisense oligonucleotide of claim 5, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of nucleobases are partially or fully deuterated.

7. The deuterated SMAD7 antisense oligonucleotide of claim 1, further comprising a plurality of riboses or deoxyriboses, wherein one or more riboses or deoxyriboses of the plurality of riboses or deoxyriboses are deuterated.

8. The deuterated SMAD7 antisense oligonucleotide of claim 7, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of riboses or deoxyriboses of the plurality of deoxyriboses are partially or fully deuterated.

9. The deuterated SMAD7 antisense oligonucleotide of claim 1, wherein the deuterated SMAD7 antisense oligonucleotide targets a region of 10 or more, 12 or more, 14 or more, 16 or more, 18 or more, 20 or more, 22 or more, 24 or more, 26 or more, 28 or more or 30 or more consecutive nucleotides of human SMAD7 (SEQ ID NO: 1).

10. The deuterated SMAD7 antisense oligonucleotide of claim 1, wherein the deuterated SMAD7 antisense oligonucleotide targets region 108-128 of human SMAD7 (SEQ ID NO: 1) (CDS of NM 005904.3).

11. The deuterated SMAD7 antisense oligonucleotide of claim 1, wherein the deuterated SMAD7 antisense oligonucleotide targets nucleotides 403, 233, 294, 295, 296, 298, 299 or 533 of human SMAD7 (SEQ ID NO: 1).

12. The deuterated SMAD7 antisense oligonucleotide of claim 1, wherein the deuterated SMAD7 antisense oligonucleotide comprises the nucleotide sequence of SEQ ID NO: 2 (5′-GTCGCCCCTTCTCCCCGCAG-3′)

13. The deuterated SMAD7 antisense oligonucleotide of claim 1, wherein the deuterated SMAD7 antisense oligonucleotide comprises the nucleotide sequence of SEQ ID NO: 3 (5′-GTCGCCCCTTCTCCCCGCAGC-3′).

14. The deuterated SMAD7 antisense oligonucleotide of claim 13, wherein at least one internucleoside linkage is a phosphorothioate linkage.

15. The deuterated SMAD7 antisense oligonucleotide of claim 14, wherein all internucleoside linkages are phosphorothioate linkages.

16. The deuterated SMAD7 antisense oligonucleotide of claim 13, wherein 2′-deoxyribonucleotides are replaced by corresponding ribonucleotides.

17. The deuterated SMAD7 antisense oligonucleotide of claim 1, wherein the deuterated SMAD7 antisense oligonucleotide is an antisense oligonucleotide phosphorothioate against SMAD7 comprising the following sequence: 5′-GTXGCCCCTTCTCCCXGCAG-3′ (SEQ ID NO: 8) wherein X is 5-methyl-2′-deoxycytidine and wherein all internucleotide linkages are phosphorothioate linkages.

18. The deuterated SMAD7 antisense oligonucleotide of claim 1, wherein the deuterated SMAD7 antisense oligonucleotide is an antisense oligonucleotide phosphorothioate against SMAD7 comprising the following sequence: 5′-GTXGCCCCTTCTCCCXGCAGC-3′ (SEQ ID NO: 9) wherein X is 5-methyl-2′-deoxycytidine and wherein the internucleotide linkages are phosphorothioate linkages.

19. The deuterated SMAD7 antisense oligonucleotide of claim 1, wherein the SMAD7 antisense oligonucleotide comprises Formula (I). The following structure of Formula (I) is drawn over four pages: 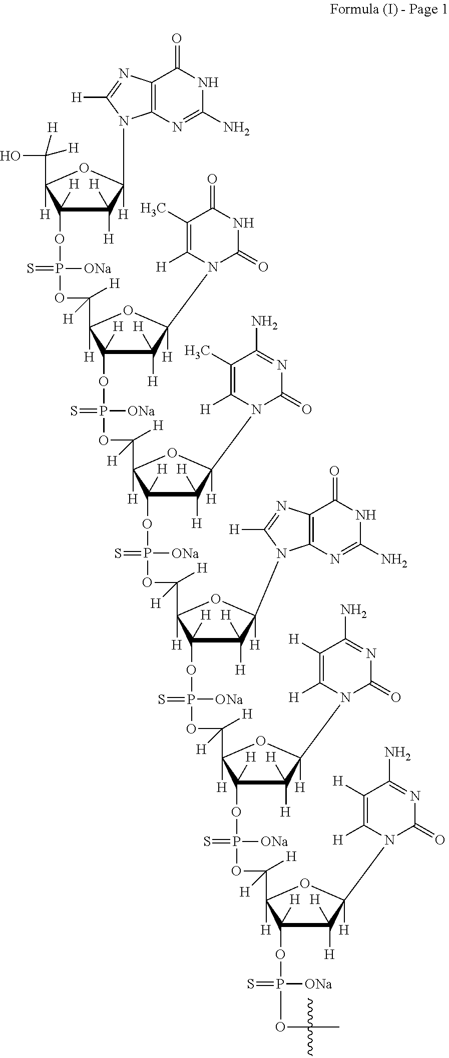 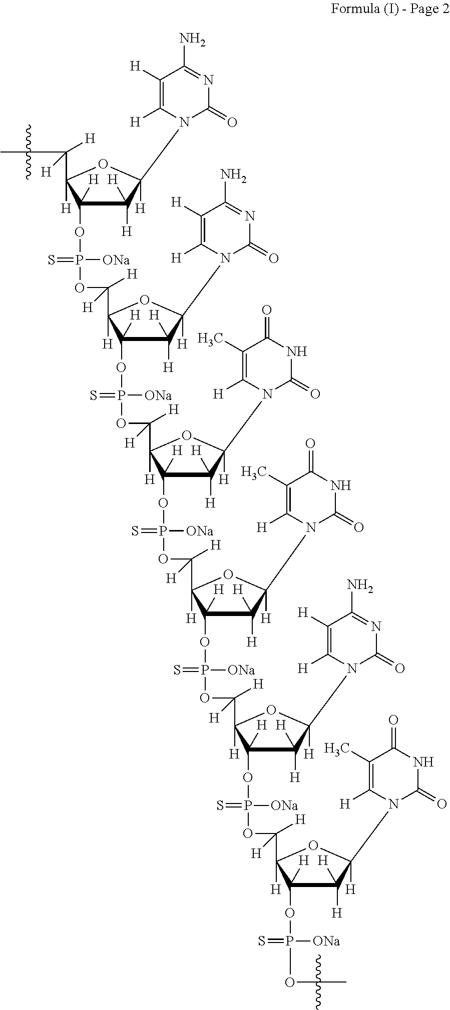 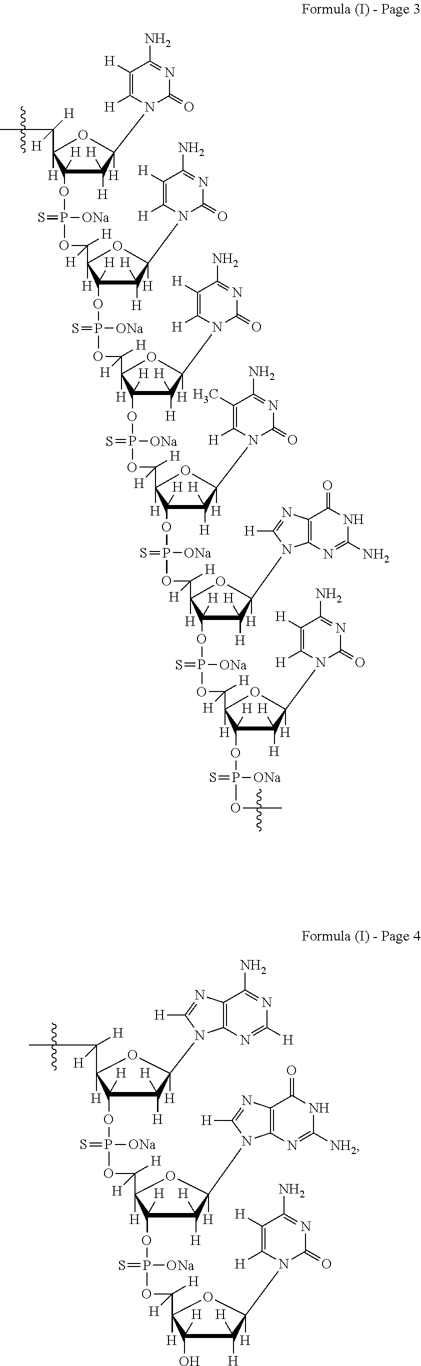
or a pharmaceutically acceptable salt or solvent thereof.

20. The deuterated SMAD7 antisense oligonucleotide of claim 19, wherein the deuterated SMAD7 antisense oligonucleotide comprises a plurality of deuterated SMAD7 antisense oligonucleotides.

21. The deuterated SMAD7 antisense oligonucleotide of any one of claims 1-20, wherein the deuterated SMAD7 antisense oligonucleotide is a pharmaceutically acceptable salt or solvent.

22. The deuterated SMAD7 antisense oligonucleotide of claim 1, wherein the SMAD7 antisense oligonucleotide comprises Formula (II). The following structure of Formula (II) is drawn over four pages: 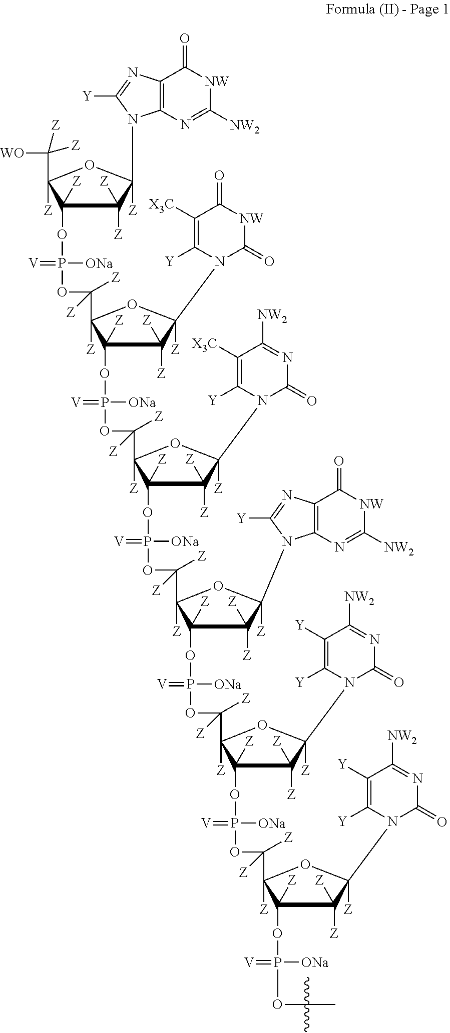 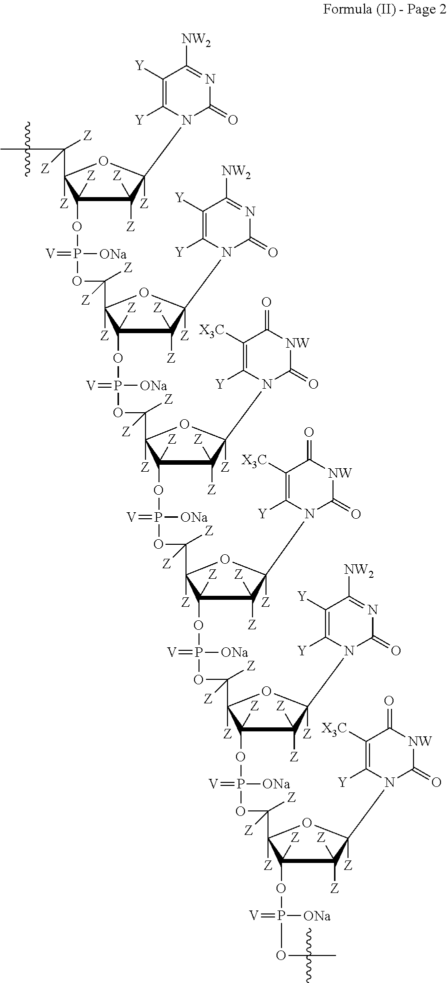 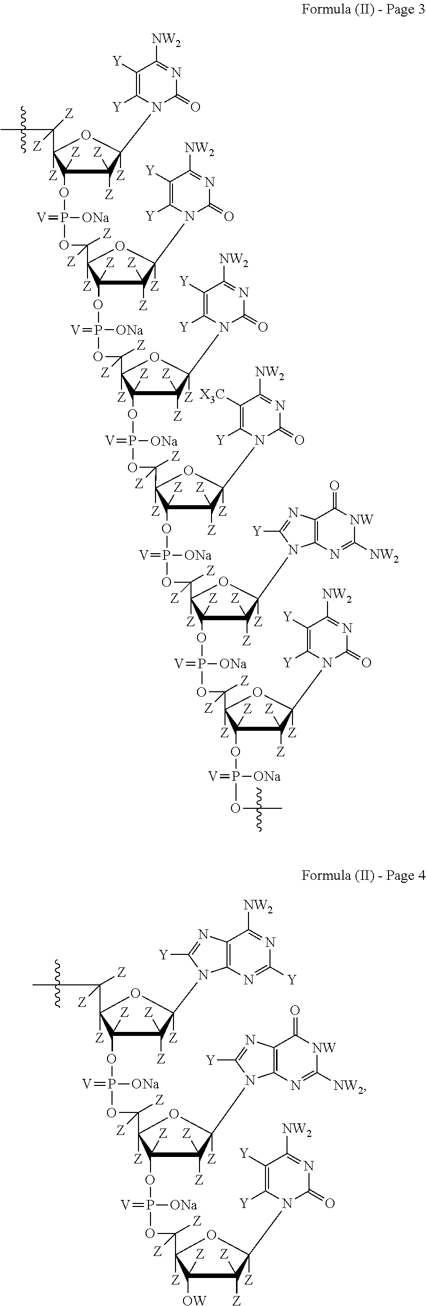
or a pharmaceutically acceptable salt or solvate thereof, wherein V is oxygen or sulfur and W, X, Y, and Z are hydrogen or deuterium.

23. The deuterated SMAD7 antisense oligonucleotide of claim 22, wherein V is oxygen.

24. The deuterated SMAD7 antisense oligonucleotide of claim 22, wherein V is sulfur.

25. The deuterated SMAD7 antisense oligonucleotide of claim 22, wherein more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, or more than 97% of W are deuterium.

26. The deuterated SMAD7 antisense oligonucleotide of claim 22, wherein more than 1%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of X are deuterium.

27. The deuterated SMAD7 antisense oligonucleotide of claim 22, wherein more than 1%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, or more than 95% of Y are deuterium.

28. The deuterated SMAD7 antisense oligonucleotide of claim 22, wherein more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 97%, more than 98%, or more than 99% of Z are deuterium.

29. The deuterated SMAD7 antisense oligonucleotide of claim 22, wherein about the same fraction of W, X, Y and Z in Formula (II) are D.

30. The deuterated SMAD7 antisense oligonucleotide of claim 22, wherein the fractions of W, X, Y and Z in Formula (II) that are D are different.

31. The deuterated SMAD7 antisense oligonucleotide of claim 22, wherein the relative sizes of the fractions of W, X, Y and Z of Formula (II) that are D has a relationship according to Table 1.

32. The deuterated SMAD7 antisense oligonucleotide of claim 22, wherein the degree of deuteration of any one deuterated W of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%. In some embodiments, the degree of deuteration of any one deuterated W of Formula (II) is 100%.

33. The deuterated SMAD7 antisense oligonucleotide of claim 22, wherein the degree of deuteration of different deuterated W of Formula (II) is about the same.

34. The deuterated SMAD7 antisense oligonucleotide of claim 22, wherein the degree of deuteration of different deuterated W of Formula (II) is different.

35. The deuterated SMAD7 antisense oligonucleotide of claim 22, wherein the degree of deuteration of only one deuterated X of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

36. The deuterated SMAD7 antisense oligonucleotide of claim 22, wherein the degree of deuteration of any one deuterated X of Formula (II) is 100%.

37. The deuterated SMAD7 antisense oligonucleotide of claim 22, wherein the degree of deuteration of only one deuterated X of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

38. The deuterated SMAD7 antisense oligonucleotide of claim 22, wherein the degree of deuteration of different deuterated X of Formula (II) is about the same.

39. The deuterated SMAD7 antisense oligonucleotide of claim 22, wherein the degree of deuteration of different deuterated X of Formula (II) is different.

40. The deuterated SMAD7 antisense oligonucleotide of claim 22, wherein the degree of deuteration of only one deuterated Y of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

41. The deuterated SMAD7 antisense oligonucleotide of claim 22, wherein the degree of deuteration of any one deuterated Y of Formula (II) is 100%.

42. The deuterated SMAD7 antisense oligonucleotide of claim 22, wherein the degree of deuteration of only one deuterated Z of Formula (II) is more than 1% more than 3%, more than 5%, more than 5%, more than 10%, more than 15%, more than 20%, more than 25%, more than 30%, more than 35%, more than 40%, more than 50%, more than 55%, more than 60%, more than 65%, more than 70%, more than 75%, more than 80%, more than 85%, more than 90%, more than 95%, more than 98%, or more than 99%.

43. The deuterated SMAD7 antisense oligonucleotide of claim 22, wherein the degree of deuteration of any one deuterated Z of Formula (II) is 100%.

44. The deuterated SMAD7 antisense oligonucleotide of claim 22, wherein the degree of deuteration of different deuterated Z of Formula (II) is about the same.

45. The deuterated SMAD7 antisense oligonucleotide of claim 22, wherein the degree of deuteration of different deuterated Z of Formula (II) is different.

46. The deuterated SMAD7 antisense oligonucleotide of claim 22, wherein the average, median, or mean degree of deuteration for the deuterated W, X, Y and Z in Formula (II) is be about the same.

47. The deuterated SMAD7 antisense oligonucleotide of claim 22, wherein the average, median or mean degree of deuteration is different for deuterated W, X, Y and Z of Formula (II).

48. The deuterated SMAD7 antisense oligonucleotide of claim 22, wherein the relative average, median or mean degrees of deuteration of deuterated W, X, Y, and Z of Formula (II) has a relationship according to Table 1.

49. The deuterated SMAD7 antisense oligonucleotide of claim 22, wherein the deuterated SMAD7 antisense oligonucleotide is a pharmaceutically acceptable salt.

50. The deuterated SMAD7 antisense oligonucleotide of claim 22, wherein the deuterated SMAD7 antisense oligonucleotide is a sodium salt.

51. The deuterated SMAD7 antisense olignucleotide of claim 1, wherein replacing the one or more hydrogens by deuterium (D) creates one or more chiral centers.

52. The deuterated SMAD7 antisense oligonucleotide of claim 51, wherein the one or more chiral centers comprise one or more C2′ or C5′ atoms in a ribose or deoxyribose in the deuterated SMAD7 antisense oligonucleotide.

53. The deuterated SMAD7 antisense oligonucleotide of claim 52, wherein more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 75%, more than 80%, more than 85%, more than 90% or more than 95% or C2′ and/or C5′ of a D-ribose or D-deoxyribose in a deuterated SMAD7 antisense oligonucleotide are chiral centers.

54. The deuterated SMAD7 antisense oligonucleotide of claim 51, wherein the deuterated SMAD7 antisense oligonucleotide comprises a racemic mixture of deuterated SMAD7 antisense oligonucleotides.

55. The deuterated SMAD7 antisense oligonucleotide of claim 51, wherein the deuterated SMAD 7 antisense oligoucleotide comprises an enantiomeric excess.

56. The deuterated SMAD7 antisense oligonucleotide of claim 51, wherein the deuterated SMAD7 antisense oligonucleotide comprises a mixture of diastereomers.

57. The deuterated SMAD7 antisense oligonucleotide of claim 51, wherein the deuterated SMAD7 antisense oligonucleotide includes a SMAD7 antisense oligonucleotide of Formula (III). The following structure of Formula (III) is drawn over four pages: 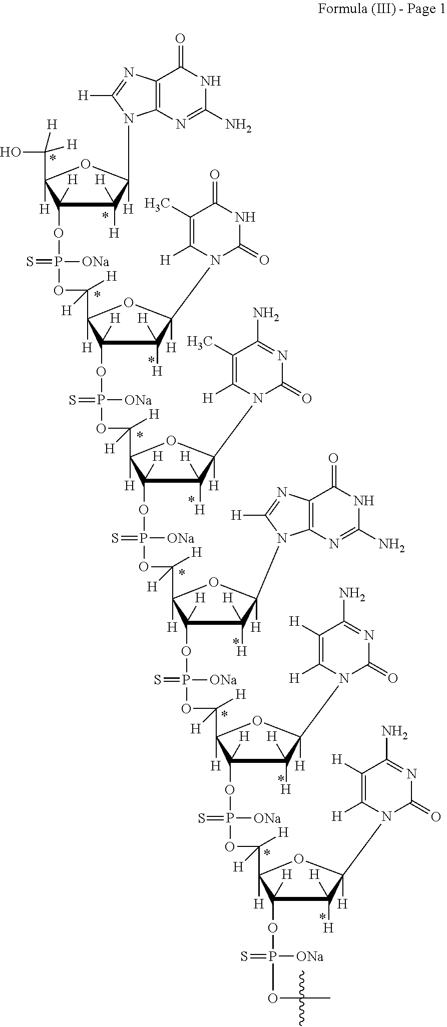 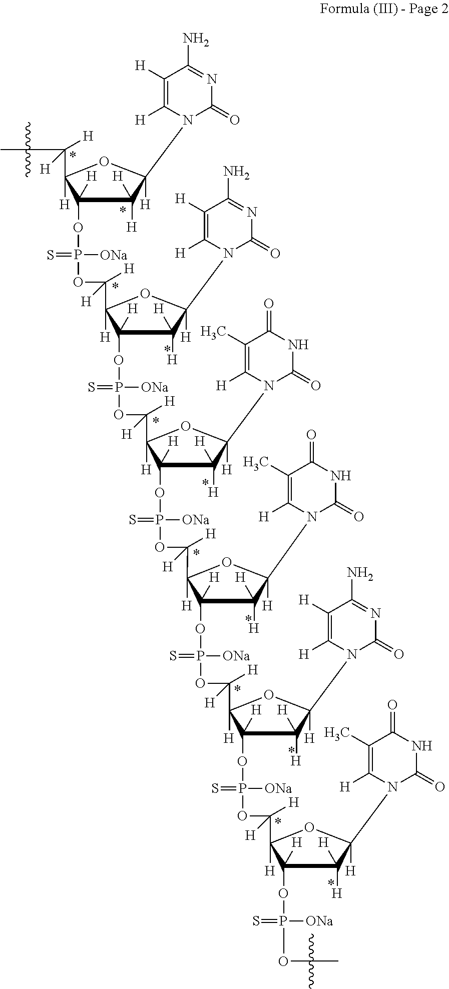 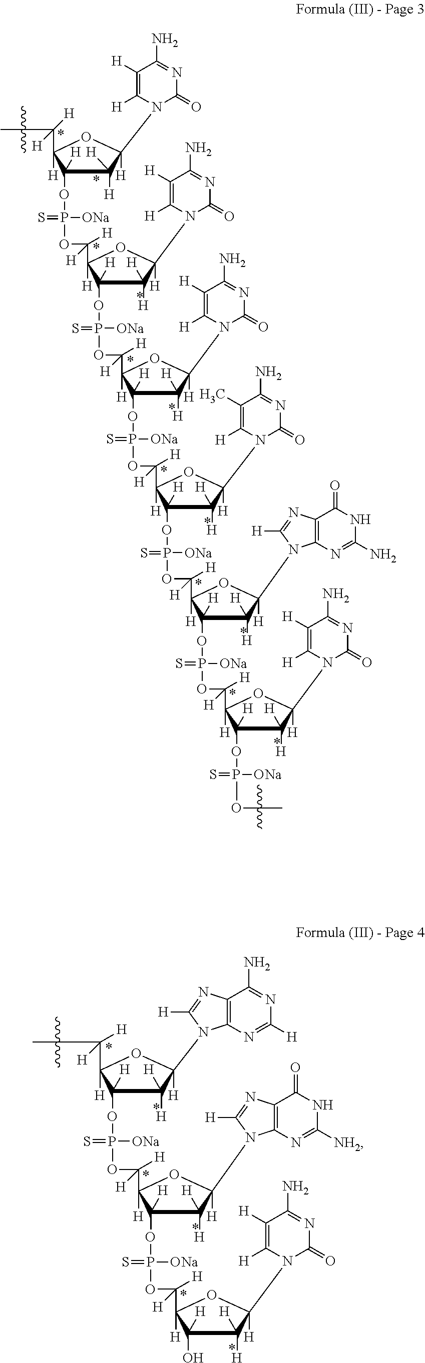
or a pharmaceutically acceptable salt or solvate thereof, wherein one or more H are replaced by D.

58. The deuterated SMAD7 antisense oligonucleotide of claim 1, wherein essentially all D in the deuterated SMAD7 antisense oligonucleotide are present in one or more nucleotides of interest.

59. The deuterated SMAD7 antisense oligonucleotide of claim 1, wherein essentially all D in the deuterated SMAD7 antisense oligonucleotide are present in one or more nucleobases of interest.

60. The deuterated SMAD7 antisense oligonucleotide of claim 59, wherein the nucleobase comprises a purine.

61. The deuterated SMAD7 antisense oligonucleotide of claim 59, wherein the nucleobase comprise a pyrimidine.

62. The deuterated SMAD7 antisense oligonucleotide of claim 59, wherein the one or more nucleobases of interest are selected from the group consisting of adenine, guanine, cytosine, thymine and uracil.

63. The deuterated SMAD7 antisense oligonucleotide of claim 1, wherein essentially all D in the deuterated SMAD7 antisense oligonucleotides are present in one or more sugar moieties of interest.

64. The deuterated SMAD7 antisense oligonucleotides of claim 63, wherein the one or more sugar moieties of interest are a ribose or deoxyribose moiety.

65. The deuterated SMAD7 antisense oligonucleotides of claim 1, wherein one or more nucleotides of interest in the deuterated SMAD7 antisense oligonucleotides comprise one or more D.

66. The deuterated SMAD7 antisense oligonucleotide of claim 65, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of the nucleotides of interest comprise one or more D.

67. The deuterated SMAD7 antisense oligonucleotide of claim 65, wherein at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 nucleotides of interest comprise one or more D.

68. The deuterated SAMD7 antisense oligonucleotide of claim 65, wherein one or more nucleotides of interest each comprise at least 1, at least 2, at least 3, at least 4, at least 5, at least 6, at least 7, at least 8, at least 9, at least 10, at least 11 or at least 12 D.

69. The deuterated SMAD7 antisense oligonucleotide of claim 65, wherein each of the one or more nucleotides of interest comprises the same number of D.

70. The deuterated SMAD7 antisense oligonucleotide of claim 65, wherein two or more nucleotides of interest comprise different numbers of D.

71. The deuterated SMAD7 antisense oligonucleotide of claim 1, wherein one or more nucleobases of interest comprise one or more D.

72. The deuterated SMAD7 antisense oligonucleotide of claim 71, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of nucleobases of interest comprise one or more D.

73. The deuterated SMAD7 antisense oligonucleotide of claim 71, wherein at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 nucleotides of interest comprise one or more D.

74. The deuterated SMAD7 antisense oligonucleotide of claim 71, wherein each of the one or more nucleobases of interest comprises at least 1, at least 2, at least 3, at least 4, or at least 5 D.

75. The deuterated SMAD7 antisense oligonucleotide of claim 74, wherein each of the nucleobases of interest comprises the same number of D.

76. The deuterated SMAD7 antisense oligonucleotide of claim 74, wherein two or more nucleobases of interest comprise different numbers of D.

77. The deuterated SMAD7 antisense oligonucleotide of claim 71, wherein the one or more nucleobases of interest comprise a purine.

78. The deuterated SMAD7 antisense oligonucleotide of claim 71, wherein the purine is an adenine or a guanine.

79. The deuterated SMAD7 antisense oligonucleotide of claim 71, wherein the one or more nucleobases of interest comprise a pyrimidine.

80. The deuterated SMAD7 antisense oligonucleotide of claim 71, wherein the pyrimidine is a cytosine, thymine or uracil.

81. The deuterated SMAD7 antisense oligonucleotide of claim 1, wherein one or more sugar moieties of interest comprise one or more D.

82. The deuterated SMAD7 antisense oligonucleotide of claim 81, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100% of sugar moieties of interest comprise one or more D.

83. The deuterated SMAD7 antisense oligonucleotide of claim 81, wherein at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 sugar moieties of interest comprise one or more D.

84. The deuterated SMAD7 antisense oligonucleotide of claim 81, wherein each of the one or more sugar moieties of interest comprises at least 1, at least 2, at least 3, at least 4, or at least 5 D.

85. The deuterated SMAD7 antisense oligonucleotide of claim 81, wherein each of the sugar moieties of interest comprises the same number of D.

86. The deuterated SMAD7 antisense oligonucleotide of claim 81, wherein two or more sugar moieties of interest comprise different numbers of D.

87. The deuterated SMAD7 antisense oligonucleotide of claim 81, wherein the one or more sugar moieties of interest comprise a ribose or deoxyribose.

88. A pharmaceutical composition comprising the deuterated SMAD7 antisense oligonucleotide of any one of claims 1-87 and a pharmaceutically acceptable adjuvant and/or excipient.

89. The pharmaceutical composition of claim 88, wherein the pharmaceutical composition is an oral pharmaceutical composition.

90. A method of treating inflammatory bowel disease (IBD), comprising administering to a patient in need thereof an effective amount of the deuterated SMAD7 antisense oligonucleotide of any one of claims 1-87, wherein the deuterated SMAD7 antisense oligonucleotide is effective to treat IBD

91. The method of claim 90, wherein the IBD is Crohn's disease.

92. The method of claim 90, wherein the IBD is ulcerative colitis.

93. The method of claim 90, wherein the antisense oligonucleotide is administered orally.
